(19) United States
(12) Patent Application Publication
(10) Pub. No.: US 2004/0242886 A1
(43) Pub. Date: Dec. 2, 2004

(54) Monocyclic diazodioxide based Bcl-2 protein antagonists related applications

(76) Inventors: Sandeep Gupta, Concord, OH (US); David Michael Carrig, Painesville, OH (US); Prabhavathi Fernandes, Lawrenceville, NJ (US)

Correspondence Address: FISH & RICHARDSON PC, 225 FRANKLIN ST, BOSTON, MA, 02110, US

(21) Appl. No.: 10/836,912

(22) Filed: Apr. 30, 2004

Related U.S. Application Data
(60) Provisional application No. 60/466,203, filed on Apr. 30, 2003.

(51) Int. Cl. C07D 471/02; C07D 231/56

(52) U.S. Cl. 546/117; 548/361.1

(57) ABSTRACT

Compounds and compositions containing compounds given by the structural Formula 8, for tumor therapeutic applications are disclosed.

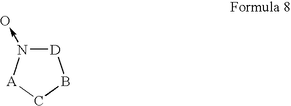

A and B are each independently selected from

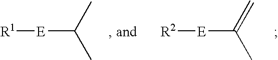

C is selected from

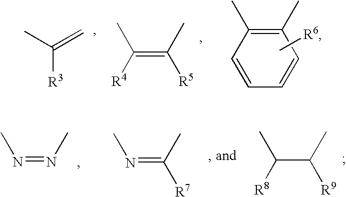

D is selected from —N—, —NO—, —NR¹⁰, —CR¹¹R¹²—, —CR¹³—, —S—, —SO—, and —SO₂—;

E is selected from single bond, —CR¹⁴R¹⁵, —NR¹⁶, —O—, —S—, —SO—, and —SO₂;

R¹ to R⁵, and R⁷ to R¹⁶ are appropriately selected to optimize physicochemical and/or biological properties. These compounds are expected to induce apoptosis in tumor cells mediated through Bcl-2 family of proteins.

### RELATED APPLICATIONS

Priority is claimed herein to U.S. provisional patent application No. 60/466,203, to Gupta et al., filed Apr. 30, 2003, entitled “Novel Monocyclic Diazodioxide based Bcl-2 Protein Antagonists and Use Thereof”. The disclosure of the above-referenced application is incorporated by reference herein in its entirety.

### FIELD

Provided herein are compositions for cancer therapy. More particularly, provided herein are monocyclic N-oxide and S-oxide derivatives and their use in cancer therapy mediated by Bcl-2 family of proteins.

### BACKGROUND

One of the most promising, but challenging approaches for the treatment of cancer involves the selective induction of apoptosis (controlled cell death and disposal) in tumor cells. Apoptosis plays an important role not only in normal cell growth and maintenance, but also in defending the organism against pathogenic microorganisms. Also, multicellular organisms use apoptotic process to destroy damaged DNA before it induces cancerous transformation. It is well recognized that one of the causes of cancer is the perturbation of the intricate balance (homeostasis) between growth and death, and that faulty regulation of apoptotic process has been implicated in many diseases including cancer, degenerative disorders and vascular diseases (Gross, A. et al., Genes Devel. 1991, 13, 1899-1911; Hawkins, C. J.; Vaux, D. L., Semin. Immunol. 1997, 9, 25-33). There are two major challenges in treating tumors using apoptotic process: first, many cancer cells have a mechanism to evade the apoptotic process; and second, the treatment protocol requires delicate balance between growth and death of normal versus cancerous cells, for too much activation of apoptotic process will result in death of normal cells and too little activation or inactivation may cause proliferation of cancer cells.

Over the past several decades, many apoptotic regulators have been identified, which include Bcl-2 (B-cell lymphoma) family of proteins. The Bcl-2 family comprises both anti-apoptotic proteins such as Bcl-2 itself, BCl-X_{L}, Bcl-w, Mcl, and A1; and pro-apoptotic proteins such as Bax, Bak, Bad, Bik, Bid, and Bok (Adams, J. M. and Cory, S., Science 1998, 281, 1322-1326). The direct link of BCL2 gene to apoptosis and cancer emerged when this key gene in follicular lymphoma was found to inhibit cell death rather than promote proliferation.

In humans, 24 members of the Bcl-2 group of proteins have been identified. These proteins are the central regulators of the intrinsic apoptotic pathway and they regulate integrity of mitochondrial membrane. Changes in the permeability or destruction of the mitochondrial membranes leads to the release of cytochrome-C and other apoptotic proteins that in concert with apoptotic protein activating factor, Apafl, carry out the activation of the initiator caspase 9. Caspases are a key group of intracellular cysteine activated-aspartate specific proteases (11 members identified in humans) which are present as inactive precursors—but upon activation, produce a cascade of proteolytic events leading to cell death. Once the initiator caspase is activated, it processes others that begin to degrade a multitude of cellular proteins signaling the initiation of the apoptotic process.

Bcl-2 itself is a 26 kilodalton protein and is related to the other members of the group by the presence of highly conserved homology domains (BH1-BH4). The pro-survival group of proteins such as Bcl-2 and BCl-X_{L} carry all the BH1-BH4 homology domains while the Bax family of pro-apoptotic proteins are characterized by the presence of BH1-BH3. The last group of proteins, the BH3 only proteins such as Bid, Bim, Bik, and Bad, are considered to be sentinel proteins responsible for triggering apoptosis in response to apoptotic signal. Both Bcl-2 and BCl-X_{L} are over-expressed in several type of tumors, including 70% of breast cancers, 80% of B-cell lymphomas, 30-60% of prostate cancers, and 90% of colorectal adenocarcinomas (Buolamvini, J. K., Curr. Opin. Chem. Biol. 1991, 3, 500-509). Over-expression of Bcl-2 and BC1-X_{L} results in blocking of apoptotic signals that leads to cell proliferation. Although the precise mechanism of action of Bcl-2 is not clearly understood, it is believed that the anti-apoptotic Bcl-2 proteins prevent the release of pro-apoptotic factors such as cytochrome-C from mitrochondria, thereby inhibiting the initiation of activities of a group of proteolytic enzymes that actually causes cell destruction (Kelekar, A. and Thompson, B., Trends Cell Biol. 1998, 8, 324-330). It has also been shown that Bcl-2 is a mitochondrial membrane-bound protein that maintains the integrity of mitrochondria and its dissociation from the membrane causes degradation of mitochondiral membrane and thereby releasing proteolytic enzyme from the mitochondrion (Cory, S. and Adams, J. M., Nature Reviews, Cancer 2002, 2, 647). The levels of Bcl-2 proteins have been shown to correlate with the resistance to many chemotherapeutic drugs and radiation therapy, and that the suppression of their activity and/or their levels restores the sensitivity to the aforementioned therapeutic agents (Reed, J. C., Adv. Pharmacol 1997, 41, 501-553). In essence, conventional cytotoxic therapy indirectly induces apoptosis through the intrinsic pathway, but cancer cells often show diminished response to such therapies. A better response, however, can be elicited by direct induction of apoptosis using processes such as impairing the action or expression of Bcl-2 like proteins or identifying compounds that mimic the BH3 only proteins.

The activities of Bcl-2 and Bcl-X_{L} are intimately connected to their binding at the BH3 region of the pro-apoptotic proteins Bax, Bak, Bid, and Bad, and the formation of such heterodimeric complex between pro- and anti-apoptotic proteins has been shown to induce apoptosis and suppression of tumor growth in animal model systems (Wang, J. L. et al., Proc. Natl. Acad. Sci. U.S.A. 2000, 97, 7124-7129). There is ample evidence in the form of NMR and X-ray data supporting the protein-protein interaction leading to the formation of heterodimers. For example the data suggests that the Bak peptide in the complex adopts an amphipathic a helical structure (BH3 domain) that interacts with Bcl-X_{L} through hydrophobic and electrostatic interactions. Mutations in Bak that prevent these interactions inhibit the ability of Bak to form the heterodimer with BCl-X_{L}. In essence, the pro-apoptotic protein sequesters the anti-apoptotic protein and, thus, it is reasonable to expect that the development of small molecule antagonists of Bcl-2 or Bcl-X_{L} present viable and attractive targets for cancer chemotherapy. Their natural survival function can be eliminated using strategies such as turning off the gene transcription, use of antisense oligonucleotides to inactivate mRNA, or directly modifying protein activity using small molecule therapeutics.

Accordingly, there has been considerable effort in developing small molecules directed at not only perturbing the protein-protein interaction, but also inhibiting the gene expression of anti-apoptotic proteins (Enyedy, I. J. et al., J. Med. Chem. 2001, 44, 4313-4324; Wang, S. and Carroll, P. G., PCT Application 2002, WO 02/097053 A2; Zeigler, A. et al., J. Natl. Cancer. Inst. 1997, 89, 1027-1036). These small molecules (Table 1) include both natural products such as gossypol (1) and antimycin (2) and synthetic compounds 3-6. The efficacy of a drug substance depends not only on the strength of the binding of these molecules to the cellular components, but also equally importantly on pharmacokinetic and pharmacodynamic parameters, including cell permeability, metabolism, serum protein binding, and the like. Most of the compounds screened thus far, including those listed in Table 1, exhibited only a modest activity, both with respect to Bcl-2 protein binding as well as cytotoxicity. Although compound 3 was disclosed as a potential anticancer compound in 1983 (Bown, D., Ph.D. Thesis 1983, Massachusetts Institute of Technology), its mode of action as Bcl-2 antagonist has been demonstrated only recently (Wang, S. and Carroll, P. G., PCT Application 2002, WO 02/13833 A2). Furthermore, the structure-activity relationship (SAR) data is very limited despite its attractive feature of having a simple structure and moderate activity.

TABLE 1
Small Molecule Inhibitors of Bcl-2 and Bcl X_{L}

| | Compound | IC₅₀ (FP) | LC₅₀ (cell) | Reference |
|---|---|---|---|---|
| 1 | 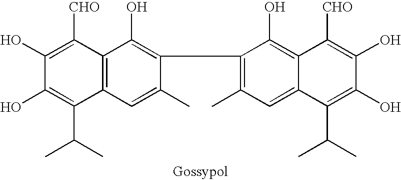 | 10 μM (Bcl-2) 0.4 μM (Bcl-X_{L}) | 1.5 μM | 10 |
| 2 | 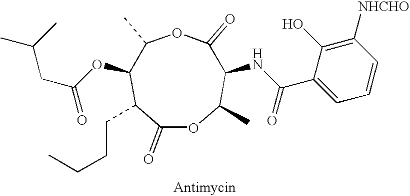 | 2.5 μM | 1.2 μM | 15 |
| 3 | 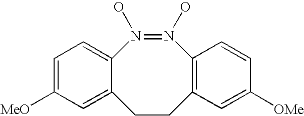 | 10 μM | 10 μM | 9,13 |
| 4 | 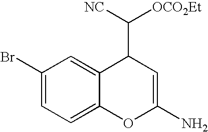 | 9 μM | 18 μM | 8,16 |
| 5 | 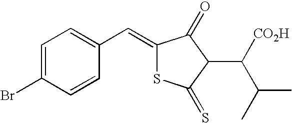 | 2.4 μM | ˜90 μM | 17 |
| 6 | 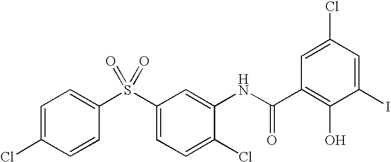 | 3.3 μM | ˜30 μM | 17 |
| 7 | 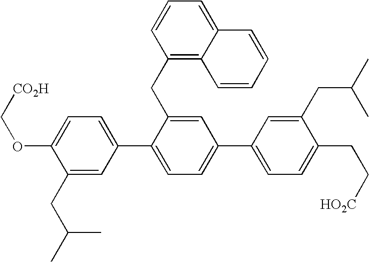 | 114 nM | Not Reported | 14 |

Most recently, de novo design of Bcl-2 antagonists by molecular modeling method yielded a potent, but highly lipophilic compound 7 with an IC₅₀ value of 114 nM (Olaf, K. et al., J. Am. Chem. Soc. 2002, 124, 11838). However, its efficacy in cell-based assay has not been established. Thus, there continues to exist the need to develop small molecule compositions having optimal Bcl-2 and BCl-X_{L} binding and pharmacological properties.

### SUMMARY

Provided herein are compounds and pharmaceutical compositions containing compounds having Formula 8:

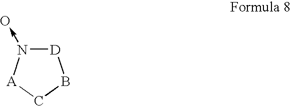

or pharmaceutically acceptable derivative sthereof, wherein A is selected from

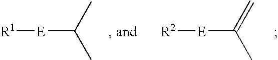

B is selected from

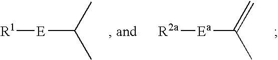

C is selected from

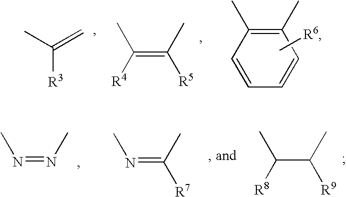

D is selected from —N—, —NO—, —NR¹⁰, —CR¹¹R¹²—, —CR¹³—, —S—, —SO—, and —SO₂—;

E and E^{a} are each independently selected from single bond, —CR¹⁴R¹⁵, —NR¹⁶, —O—, —S—, —SO—, and —SO₂;

R¹ to R⁵, R⁷ to R¹⁶ and R^{2a} are appropriately selected to optimize physicochemical and/or biological properties such as, bioavailability, pharmacokinetics, Bcl-2 activity, metabolism, etc. In certain embodiments, the compounds provided herein are selected with the proviso that when C is

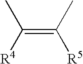

and E and E^{a} are both SO₂, then at least one of R² and R^{2a} is not CH₃.

Also of interest are any pharmaceutically-acceptable derivatives, including salts, esters, enol ethers or esters, acetals, ketals, orthoesters, hemiacetals, hemiketals, solvates, hydrates or prodrugs of the compounds. Pharmaceutically-acceptable salts, include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-parachlorobenzyl-2-pyrrolidin-1′-ylmethylbenzimidazole, diethylamine and other alkylamines, piperazine, tris(hydroxymethyl)aminomethane, alkali metal salts, such as but not limited to lithium, potassium and sodium, alkali earth metal salts, such as but not limited to barium, calcium and magnesium, transition metal salts, such as but not limited to zinc and other metal salts, such as but not limited to sodium hydrogen phosphate and disodium phosphate, and also including, but not limited to, salts of mineral acids, such as but not limited to hydrochlorides and sulfates, salts of organic acids, such as but not limited to acetates, lactates, malates, tartrates, citrates, ascorbates, succinates, butyrates, valerates and fumarates.

Pharmaceutical formulations for administration by an appropriate route and means containing effective concentrations of one or more of the compounds provided herein or pharmaceutically acceptable derivatives, such as salts, esters, enol ethers or esters, acetals, ketals, orthoesters, hemiacetals, hemiketals, solvates, hydrates or prodrugs, of the compounds that deliver amounts effective for the treatment of Bcl-2 protein-mediated disorders, are also provided. Bcl-2 protein-mediated disorders include, but are not limited to, cancers, tumors, hyperproliferative diseases, acquired immune deficiency syndrome, degenerative conditions, and vascular diseases. In certain embodiments, the cancers include, but are not limited to B-cell lymphoma including B-cell lymphoma-2, B-cell leukemia, skin cancer, pancreatic cancer, ovarian cancer, liver cancer, bladder cancer, adrenal carcinoma, breast cancer, prostate cancer, colorectal cancer including colorectal adenocarcinomas, follicular lymphoma.

The formulations are compositions suitable for administration by any desired route and include solutions, suspensions, emulsions, tablets, dispersible tablets, pills, capsules, powders, dry powders for inhalation, sustained release formulations, aerosols for nasal and respiratory delivery, patches for transdermal delivery and any other suitable route. The compositions should be suitable for oral administration, parenteral administration by injection, including subcutaneously, intramuscularly or intravenously as an injectable aqueous or oily solution or emulsion, transdermal administration and other selected routes.

Methods using such compounds and compositions for modulating the activity of a Bcl-2 protein are provided. The methods are effected by contacting a composition containing the Bcl-2 protein with one or more of the compounds or compositions.

Methods for treatment of Bcl-2 protein-mediated disorders, including, but not limited to, cancers, tumors, hyperproliferative diseases, acquired immune deficiency syndrome, degenerative conditions, and vascular diseases. In certain embodiments, the cancers include, but are not limited to B-cell lymphoma including B-cell lymphoma-2, B-cell leukemia, skin cancer, pancreatic cancer, ovarian cancer, liver cancer, bladder cancer, adrenal carcinoma, breast cancer, prostate cancer, colorectal cancer including colorectal adenocarcinomas, follicular lymphoma.

In practicing the methods, effective amounts of formulations containing therapeutically effective concentrations of the compounds formulated for oral, intravenous, local and topical application for the treatment of Bcl-2 protein-mediated diseases or disorders are administered to an individual exhibiting the symptoms of one or more of these disorders. The amounts are effective to ameliorate or eliminate one or more symptoms of the diseases or disorders.

Articles of manufacture containing packaging material, a compound provided herein, or a pharmaceutically acceptable derivative thereof, which is effective for ameliorating the symptoms of a Bcl-2 protein-mediated disorder, within the packaging material, and a label that indicates that the compound, or pharmaceutically acceptable derivative thereof, is used for ameliorating the symptoms of a Bcl-2 protein-mediated disorder are provided.

### DETAILED DESCRIPTION

A. Definitions

Unless defined otherwise, all technical and scientific terms used herein have the same meaning as is commonly understood by one of ordinary skill in the art to which this invention belongs. All patents, applications, published applications and other publications are incorporated by reference in their entirety. In the event that there are a plurality of definitions for a term herein, those in this section prevail unless stated otherwise.

As used herein, a “Bcl-2 protein” is a member of a class of proteins affecting apoptosis. The class includes at least 24 individual proteins. The proteins are both pro-apoptotic (e.g., Bax, Bak, Bad, Bik, Bid and Bok) and anti-apoptotic (e.g., Bcl-2, BCl-X_{L}, Bcl-w, Mcl and A1).

As used herein, “Bcl-2” refers to the specific protein designated Bcl-2.

As used herein, an “anti-apoptotic Bcl-2 protein” is a Bcl-2 protein whose activity prevents or delays apoptosis. Such proteins include but are not limited to Bcl-2, Bcl-XL, Bcl-w, Mcl and A1.

As used herein, a “pro-apoptotic Bcl-2 protein” is a Bcl-2 protein whose activity induces or assists apoptosis. Such proteins include but are not limited to Bax, Bak, Bad, Bik, Bid and Bok.

As used herein, pharmaceutically acceptable derivatives of a compound include salts, esters, enol ethers, enol esters, acetals, ketals, orthoesters, hemiacetals, hemiketals, solvates, hydrates or prodrugs thereof. Such derivatives may be readily prepared by those of skill in this art using known methods for such derivatization. The compounds produced may be administered to animals or humans without substantial toxic effects and either are pharmnaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating Bcl-2 protein mediated diseases or disorders, or diseases or disorders in which Bcl-2 protein activity is implicated.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, LC₅₀ refers to a concentration of a particular test compound that kills 50% of cells in an in vitro assay that measures such response, including the assays described herein.

As used herein, a prodrug is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmnacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, N.Y., pages 388-392).

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. In the case of amino acid residues, such residues may be of either the L- or D-form. The configuration for naturally occurring amino acid residues is generally L. When not specified the residue is the L form. As used herein, the term “amino acid” refers to α-amino acids which are racemic, or of either the D- or L-configuration. The designation “d” preceding an amino acid designation (e.g., dAla, dSer, dVal, etc.) refers to the D-isomer of the amino acid. The designation “d1” preceding an amino acid designation (e.g., d1Pip) refers to a mixture of the L- and D-isomers of the amino acid. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, alkyl, alkenyl and alkynyl carbon chains, if not specified, contain from 1 to 20 carbons, or 1 or 2 to 16 carbons, and are straight or branched. Alkenyl carbon chains of from 2 to 20 carbons, in certain embodiments, contain 1 to 8 double bonds and alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, allyl(propenyl) and propargyl(propynyl). As used herein, lower alkyl, lower alkenyl, and lower alkynyl refer to carbon chains having from about 1 or about 2 carbons up to about 6 carbons. As used herein, “alk(en)(yn)yl” refers to an alkyl group containing at least one double bond and at least one triple bond.

As used herein, “cycloalkyl” refers to a saturated mono- or multi-cyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or spiro-connected fashion. “Cycloalk(en)(yn)yl” refers to a cycloalkyl group containing at least one double bond and at least one triple bond.

As used herein, “aryl” refers to aromatic monocyclic or multicyclic groups containing from 6 to 19 carbon atoms. Aryl groups include, but are not limited to groups such as unsubstituted or substituted fluorenyl, unsubstituted or substituted phenyl, and unsubstituted or substituted naphthyl.

As used herein, “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl.

As used herein, “heterocyclyl” refers to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur. In embodiments where the heteroatom(s) is(are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, or the nitrogen may be quaternized to form an ammonium group where the substituents are selected as above.

As used herein, “aralkyl” refers to an alkyl group in which one of the hydrogen atoms of the alkyl is replaced by an aryl group.

As used herein, “heteroaralkyl” refers to an alkyl group in which one of the hydrogen atoms of the alkyl is replaced by a heteroaryl group.

As used herein, “halo”, “halogen” or “halide” refers to F, Cl, Br or I.

As used herein, pseudohalides or pseudohalo groups are groups that behave substantially similar to halides. Such compounds can be used in the same manner and treated in the same manner as halides. Pseudohalides include, but are not limited to, cyanide, cyanate, thiocyanate, selenocyanate, trifluoromethoxy, and azide.

As used herein, “haloalkyl” refers to an alkyl group in which one or more of the hydrogen atoms are replaced by halogen. Such groups include, but are not limited to, chloromethyl, trifluoromethyl andl-chloro-2-fluoroethyl.

As used herein, “haloalkoxy” refers to RO— in which R is a haloalkyl group.

As used herein, “sulfinyl” or “thionyl” refers to —S(O)—. As used herein, “sulfonyl” or “sulfuryl” refers to —S(O)₂—. As used herein, “sulfo” refers to —S(O)₂O—.

As used herein, “carboxy” refers to a divalent radical, —C(O)O—.

As used herein, “aminocarbonyl” refers to —C(O)NH₂.

As used herein, “alkylaminocarbonyl” refers to —C(O)NHR in which R is alkyl, including lower alkyl. As used herein, “dialkylaminocarbonyl” refers to —C(O)NR′R in which R′ and R are each independently alkyl, including lower alkyl; “carboxamide” refers to groups of formula —NR′COR in which R′ and R are each independently alkyl, including lower alkyl.

As used herein, “arylalkylaminocarbonyl” refers to —C(O)NRR′ in which one of R and R′ is aryl, including lower aryl, such as phenyl, and the other of R and R′ is alkyl, including lower alkyl.

As used herein, “arylaminocarbonyl” refers to —C(O)NHR in which R is aryl, including lower aryl, such as phenyl.

As used herein, “hydroxycarbonyl” refers to —COOH.

As used herein, “alkoxycarbonyl” refers to —C(O)OR in which R is alkyl, including lower alkyl.

As used herein, “aryloxycarbonyl” refers to —C(O)OR in which R is aryl, including lower aryl, such as phenyl.

As used herein, “alkoxy” and “alkylthio” refer to RO— and RS—, in which R is alkyl, including lower alkyl.

As used herein, “aryloxy” and “arylthio” refer to RO— and RS—, in which R is aryl, including lower aryl, such as phenyl.

As used herein, “alkylene” refers to a straight, branched or cyclic, in certain embodiments straight or branched, divalent aliphatic hydrocarbon group, in one embodiment having from 1 to about 20 carbon atoms, in another embodiment having from 1 to 12 carbons. In a further embodiment alkylene includes lower alkylene. There may be optionally inserted along the alkylene group one or more oxygen, sulfur, including S(═O) and S(═O)₂ groups, or substituted or unsubstituted nitrogen atoms, including —NR— and —N⁺RR— groups, where the nitrogen substituent(s) is(are) alkyl, aryl, aralkyl, heteroaryl, heteroaralkyl or COR′, where R′ is alkyl, aryl, aralkyl, heteroaryl, heteroaralkyl, —OY or —NYY, where Y is hydrogen, alkyl, aryl, heteroaryl, cycloalkyl or heterocyclyl. Alkylene groups include, but are not limited to, methylene (—CH₂—), ethylene (—CH₂CH₂—), propylene (—(CH₂)₃—), methylenedioxy (—O—CH₂—O—) and ethylenedioxy (—O—(CH₂)₂—O—). The term “lower alkylene” refers to alkylene groups having 1 to 6 carbons. In certain embodiments, alkylene groups are lower alkylene, including alkylene of 1 to 3 carbon atoms.

As used herein, “azaalkylene” refers to —(CRR)ₙ—NR—(CRR)ₘ—, where n and m are each independently an integer from 0 to 4. As used herein, “oxaalkylene” refers to —(CRR)ₙ—O—(CRR)ₘ—, where n and m are each independently an integer from 0 to 4. As used herein, “thiaalkylene” refers to —(CRR)ₙ—S—(CRR)ₘ—, —(CRR)ₙ—S(═O)—(CRR)ₘ—, and —(CRR)ₙ—S(═O)₂—(CRR)ₘ—, where n and m are each independently an integer from 0 to 4.

As used herein, “alkenylene” refers to a straight, branched or cyclic, in one embodiment straight or branched, divalent aliphatic hydrocarbon group, in certain embodiments having from 2 to about 20 carbon atoms and at least one double bond, in other embodiments 1 to 12 carbons. In further embodiments, alkenylene groups include lower alkenylene. There may be optionally inserted along the alkenylene group one or more oxygen, sulfur or substituted or unsubstituted nitrogen atoms, where the nitrogen substituent is alkyl. Alkenylene groups include, but are not limited to, —CH═CH—CH═CH— and —CH═CH—CH₂—. The term “lower alkenylene” refers to alkenylene groups having 2 to 6 carbons. In certain embodiments, alkenylene groups are lower alkenylene, including alkenylene of 3 to 4 carbon atoms.

As used herein, “alkynylene” refers to a straight, branched or cyclic, in certain embodiments straight or branched, divalent aliphatic hydrocarbon group, in one embodiment having from 2 to about 20 carbon atoms and at least one triple bond, in another embodiment 1 to 12 carbons. In a further embodiment, alkynylene includes lower alkynylene. There may be optionally inserted along the alkynylene group one or more oxygen, sulfur or substituted or unsubstituted nitrogen atoms, where the nitrogen substituent is alkyl. Alkynylene groups include, but are not limited to, —C≡C—C≡C—, —C≡C— and —C≡C—CH₂—. The term “lower alkynylene” refers to alkynylene groups having 2 to 6 carbons. In certain embodiments, alkynylene groups are lower alkynylene, including alkynylene of 3 to 4 carbon atoms.

As used herein, “alk(en)(yn)ylene” refers to a straight, branched or cyclic, in certain embodiments straight or branched, divalent aliphatic hydrocarbon group, in one embodiment having from 2 to about 20 carbon atoms and at least one triple bond, and at least one double bond; in another embodiment 1 to 12 carbons. In further embodiments, alk(en)(yn)ylene includes lower alk(en)(yn)ylene. There may be optionally inserted along the alkynylene group one or more oxygen, sulfur or substituted or unsubstituted nitrogen atoms, where the nitrogen substituent is alkyl. Alk(en)(yn)ylene groups include, but are not limited to, —C═C—(CH₂)ₙ—C≡C—, where n is I or 2. The term “lower alk(en)(yn)ylene” refers to alk(en)(yn)ylene groups having up to 6 carbons. In certain embodiments, alk(en)(yn)ylene groups have about 4 carbon atoms.

As used herein, “cycloalkylene” refers to a divalent saturated mono- or multicyclic ring system, in certain embodiments of 3 to 10 carbon atoms, in other embodiments 3 to 6 carbon atoms; cycloalkenylene and cycloalkynylene refer to divalent mono- or multicyclic ring systems that respectively include at least one double bond and at least one triple bond. Cycloalkenylene and cycloalkynylene groups may, in certain embodiments, contain 3 to 10 carbon atoms, with cycloalkenylene groups in certain embodiments containing 4 to 7 carbon atoms and cycloalkynylene groups in certain embodiments containing 8 to 10 carbon atoms. The ring systems of the cycloalkylene, cycloalkenylene and cycloalkynylene groups may be composed of one ring or two or more rings which may be joined together in a fused, bridged or spiro-connected fashion. “Cycloalk(en)(yn)ylene” refers to a cycloalkylene group containing at least one double bond and at least one triple bond.

As used herein, “arylene” refers to a monocyclic or polycyclic, in certain embodiments monocyclic, divalent aromatic group, in one embodiment having from 5 to about 20 carbon atoms and at least one aromatic ring, in another embodiment 5 to 12 carbons. In further embodiments, arylene includes lower arylene. Arylene groups include, but are not limited to, 1,2-, 1,3- and 1,4-phenylene. The term “lower arylene” refers to arylene groups having 6 carbons.

As used herein, “heteroarylene” refers to a divalent monocyclic or multicyclic aromatic ring system, in one embodiment of about 5 to about 15 atoms in the ring(s), where one or more, in certain embodiments 1 to 3, of the atoms in the ring system is a heteroatom, that is, an element other than carbon, including but not limited to, nitrogen, oxygen or sulfur. The term “lower heteroarylene” refers to heteroarylene groups having 5 or 6 atoms in the ring.

As used herein, “heterocyclylene” refers to a divalent monocyclic or multicyclic non-aromatic ring system, in certain embodiments of 3 to 10 members, in one embodiment 4 to 7 members, in another embodiment 5 to 6 members, where one or more, including 1 to 3, of the atoms in the ring system is a heteroatom, that is, an element other than carbon, including but not limited to, nitrogen, oxygen or sulfur.

As used herein, “substituted alkyl,” “substituted alkenyl,” “substituted alkynyl,” “substituted cycloalkyl,” “substituted cycloalkenyl,” “substituted cycloalkynyl,” “substituted aryl,” “substituted heteroaryl,” “substituted heterocyclyl,” “substituted alkylene,” “substituted alkenylene,” “substituted alkynylene,” “substituted cycloalkylene,” “substituted cycloalkenylene,” “substituted cycloalkynylene,” “substituted arylene,” “substituted heteroarylene” and “substituted heterocyclylene” refer to alkyl, alkenyl, alkynyl, cycloalkyl, cycloalkenyl, cycloalkynyl, aryl, heteroaryl, heterocyclyl, alkylene, alkenylene, alkynylene, cycloalkylene, cycloalkenylene, cycloalkynylene, arylene, heteroarylene and heterocyclylene groups, respectively, that are substituted with one or more substituents, in certain embodiments one, two, three or four substituents, where the substituents are as defined herein, in one embodiment selected from Q¹.

As used herein, “alkylidene” refers to a divalent group, such as ═CR′R″, which is attached to one atom of another group, forming a double bond. Alkylidene groups include, but are not limited to, methylidene (═CH₂) and ethylidene (═CHCH₃). As used herein, “arylalkylidene” refers to an alkylidene group in which either R′ or R″ is an aryl group. “Cycloalkylidene” groups are those where R′ and R″ are linked to form a carbocyclic ring. “Heterocyclylidene” groups are those where at least one of R′ and R″ contain a heteroatom in the chain, and R′ and R″ are linked to form a heterocyclic ring.

As used herein, “amido” refers to the divalent group —C(O)NH—. “Thioamido” refers to the divalent group —C(S)NH—. “Oxyamido” refers to the divalent group —OC(O)NH—. “Thiaamido” refers to the divalent group —SC(O)NH—. “Dithiaamido” refers to the divalent group —SC(S)NH—. “Ureido” refers to the divalent group —HNC(O)NH—. “Thioureido” refers to the divalent group —HNC(S)NH—.

As used herein, “semicarbazide” refers to —NHC(O)NHNH—. “Carbazate” refers to the divalent group —OC(O)NHNH—. “Isothiocarbazate” refers to the divalent group —SC(O)NHNH—. “Thiocarbazate” refers to the divalent group —OC(S)NHNH—. “Sulfonylhydrazide” refers to the divalent group —SO₂NHNH—. “Hydrazide” refers to the divalent group —C(O)NHNH—. “Azo” refers to the divalent group —N═N—. “Hydrazinyl” refers to the divalent group —NH—NH—.

Where the number of any given substituent is not specified (e.g., haloalkyl), there may be one or more substituents present. For example, “haloalkyl” may include one or more of the same or different halogens. As another example, “C₁₋₃ alkoxyphenyl” may include one or more of the same or different alkoxy groups containing one, two or three carbons.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

B. Compounds

Provided herein are compounds and pharmaceutical compositions containing compounds of Formula 8

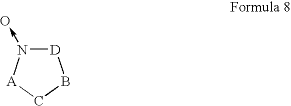

or a pharmaceutically acceptable derivative thereof, wherein

A and B are independently selected from the group consisting of

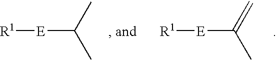

C is selected from the group consisting of

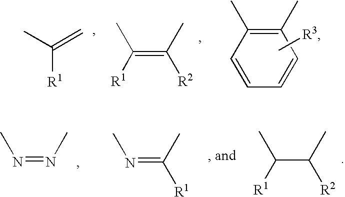

D is selected from the group consisting of —N—, —NO—, —NR⁴, —CR¹R²—, —CR¹—, —S—, —SO—, and —SO₂—. E is selected from the group consisting of single bond, —CR¹R², —NR⁴, —O—, —S—, —SO—, and —SO₂. R¹ R², and R⁴ are independently selected from the group consisting of hydrogen, C1-C10 alkyl, C3-C10 cycloalkyl, C1-C10 acyl, C₅-C₂₀ aryl unsubstituted or substituted with electron withdrawing groups including, but not limited to halogen, trihalomethyl, cyano, nitro, or alkoxycarbonyl, or electron donating groups including, but not limited to hydroxyl, alkoxyl, acyloxyl, amino, alkylamino, acylamino, mercapto, and alkylthio, C₅-C₂₀ heteroaryl unsubstituted or substituted with electron withdrawing groups including, but not limited to halogen, trihalomethyl, cyano, nitro, or alkoxycarbonyl, or electron donating groups including, but not limited to hydroxyl, alkoxyl, acyloxyl, amino, alkylamino, acylamino, mercapto, and alkylthio, C1-C10 alkoxyl, C1-C10 alkoxyalkyl, amino, C1-C10 aminoalkyl, C1-C10 akylaminoalkyl, hydroxyl, C1-C10 hydroxyalkyl, carboxyl, C1-C10 carboxyalkyl, C1-C10 alkoxylcarbonyl, C1-C10 alkoxycarbonlyalkyl, halogen, mono- or polyhaloalkyl, mono- or polyhaloalkoxyl, cyano, nitro, mercapto, C1-C10 mercaptoalkyl, C1-C10 thioalkyl, C1-C10 alkylthioalkyl, C1-C10 sulfonylalkyl, and C1-C10 alkylsulfonylalkyl. R³ is selected from the group consisting of hydrogen; electron donating groups such as C1-C 10 alkyl, C3-C10 cycloalkyl, hydroxyl, C1-C10 alkoxyl, amino, C1-C10 acylamino, mercapto, and C1-C10 alkylthio; electron withdrawing groups such as C1-C10 acyl, halo, mono- or polyhaloalkyl, cyano, nitro, carboxyl, C1-C10 alkoxylcarbonyl, C1-C10 alkylsulfonyl; C5-C10aryl; C1-C10 alkoxyalkyl; C1-C10 aminoalkyl; C1-C10 akylaminoalkyl; C1-C10 hydroxyalkyl; C1-C10 carboxyalkyl; C1-C10 alkoxycarbonlyalkyl; C1-C10 mercaptoalkyl; C1-C10 alkylthioalkyl; C1-C10 sulfonylalkyl; and C1-C10 alkylsulfonylalkyl.

In certain embodiments, the compounds have formula 8, wherein

A is selected from

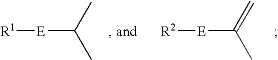

B is selected from

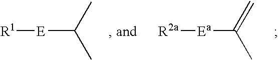

C is selected from

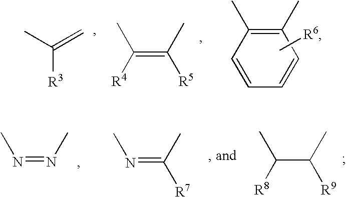

D is selected from —N—, —NO—, —NR¹⁰, —CR¹¹R¹²—, —CR¹³—, —S—, —SO—, and —SO₂—;

E and E^{a} are each independently selected from single bond, —CR¹⁴R¹⁵, —NR¹⁶, —O—, —S—, —SO—, and —SO₂;

R¹ to R⁵, R⁷ to R ¹⁶, and R^{2a} are each independently selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C1-C10 alkylsulfonylalkyl, C1-C10 alkylcarbonylaminoC1-C10alkyl, C₃-C₂₀ heterocyclyl, C₃-C₂₀ heterocyclylalkyl, C₅-C₂₀ aralkyl, C₅-C₂₀ aryl, C₅-C₂₀ heteroaralkyl and C₅-C₂₀ heteroaryl, wherein the aryl and heteroaryl groups are optionally substituted with one or more electron withdrawing groups including, but not limited to halogen, trihaloalkyl, cyano, nitro, carboxy or alkoxycarbonyl, or electron donating groups including, but not limited to hydroxyl, alkoxy, acyloxyl, amino, alkylamino, acylamino, mercapto, or alkylthio, with the proviso that when C is

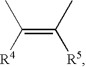

and E and E^{a} are both SO₂, then at least one of R² and R^{2a} is not CH₃;

R⁶ is selected from hydrogen; electron donating groups such as C1-C10 alkyl, C3-C10 cycloalkyl, hydroxyl, C1-C10 alkoxy, amino, C1-C10 acylamino, mercapto or C1-C10 alkylthio; and electron withdrawing groups such as C1-C10 acyl, halo, mono- or polyhaloalkyl, cyano, nitro, carboxy, C1-C10 alkoxycarbonyl, C1-C10 alkylsulfonyl, C5-C10 aryl, C1-C10 alkoxyalkyl, C1-C10 arninoalkyl, C1-C10 alkylaminoalkyl, C1-C10 hydroxyalkyl, C1-C10 carboxyalkyl, C1-C10 alkoxycarbonylalkyl, C1-C10 mercaptoalkyl, C1-C10 alkylthioalkyl, C1-C10 sulfonylalkyl, and C1-C10 alkylsulfonylalkyl.

In one embodiment, the compounds are of structural Formula 8, wherein

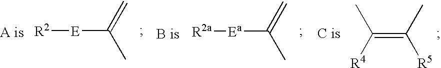

D is —N—, —NO—, or —CR¹³—;

E and E^{a} are each independently selected from a single bond, —CR¹⁴R¹⁵, —NR¹⁶, —O—, —S—, —SO—, and —SO₂;

R², R^{2a}, R⁴, R⁵ and R¹³ to R¹⁶, and R⁴ are each independently selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C1-C10 alkylsulfonylalkyl, C1-C10 alkylcarbonylaminoC1-C10alkyl, C₃-C₂₀ heterocyclyl, C₃-C₂₀ heterocyclylalkyl, C₅-C₂₀ aralkyl, C₅-C₂₀ aryl, C₅-C₂₀ heteroaralkyl and C₅-C₂₀ heteroaryl, wherein aryl and heteroaryl groups are optionally substituted with one or more electron withdrawing groups including, but not limited to halogen, trihaloalkyl, cyano, nitro, carboxy or alkoxycarbonyl, or electron donating groups including, but not limited to hydroxyl, alkoxy, acyloxyl, amino, alkylamino, acylamino, mercapto, or alkylthio, with the proviso that when E and E^{a} are both SO₂, then at least one of R² and R^{2a} is not CH₃.

In another embodiment, the compounds provided herein have Formula 8, wherein

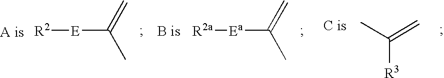

D is —NR¹⁰—; E and E^{a} are each independently selected from a single bond, —CR¹⁴R¹⁵, —NR¹⁶, —O—, —S—, —SO—, and —SO₂—; R², R^{2a}, R³, R¹⁰, R¹⁴, R¹⁵ , and R¹⁶ are each independently selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C1-C10 alkylsulfonylalkyl, C1-C10 alkylcarbonylaminoC1-C10alkyl, C₃-C₂₀ heterocyclyl, C₃-C₂₀ heterocyclylalkyl, C5-C20 aralkyl, C5-C20 aryl, C5-C20 heteroaralkyl and C5-C20 heteroaryl, wherein the aryl and heretoaryl groups are optionally substituted with one or more electron withdrawing groups including, but not limited to halogen, trihaloalkyl, cyano, nitro, carboxy or alkoxycarbonyl, or electron donating groups including, but not limited to hydroxyl, alkoxy, acyloxyl, amino, alkylamino, acylamino, mercapto, or alkylthio.

In another embodiment, the compounds have Formula 8, wherein

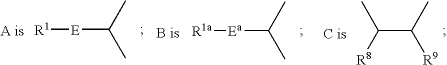

D is —CR¹¹R¹²; E and E^{a} are each selected from single bond, —CR¹⁴R¹⁵, —NR¹⁶, —O—, —S—, —SO—, and —SO₂; R¹, R^{1a}, R⁸, R⁹, R¹¹, R¹², R¹⁴, R¹⁵ and R¹⁶ are each independently selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C 10 thioalkyl, C1-C10 alkylthioalkyl, C1-C10 sulfonylalkyl, C1-C10 alkylsulfonylalkyl, , C1-C10 alkylcarbonylaminoC1-C10alkyl, C₃-C₂₀ heterocyclyl, C₃-C₂₀ heterocyclylalkyl, C5-C20 aralkyl, C5-C20 aryl, C5-C20 heteroaralkyl and C5-C20 heteroaryl, wherein the aryl and heteroaryl groups are optionally substituted with one or more electron withdrawing groups including, but not limited to halogen, trihaloalkyl, cyano, nitro, carboxy or alkoxycarbonyl, or electron donating groups including, but not limited to hydroxyl, alkoxy, acyloxyl, amino, alkylamino, acylamino, mercapto, or alkylthio.

In another embodiment, the compounds have the structural Formula 8, wherein

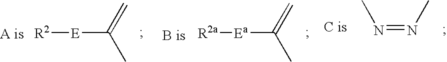

D is —N—, —NO—; E and E^{a} are each independently selected from a single bond, —O—, —S—, —SO—, and —SO₂; R² and R^{2a} are each independently selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C1-C10 alkylsulfonylalkyl, C1-C10 alkylcarbonylaminoC1-C10alkyl, C₃-C₂₀ heterocyclyl, C₃-C₂₀ heterocyclylalkyl, C₅-C₂₀ aralkyl, C₅-C₂₀ aryl, C₅-C₂₀ heteroaralkyl and C₅-C₂₀ heteroaryl, wherein the aryl and heteroaryl groups are optionally substituted with one or more electron withdrawing groups including, but not limited to halogen, trihaloalkyl, cyano, nitro, carboxy or alkoxycarbonyl, or electron donating groups including, but not limited to hydroxyl, alkoxy, acyloxyl, amino, alkylamino, acylamino, mercapto, or alkylthio.

In another embodiment, the compounds provided herein have the structural Formula 8, wherein

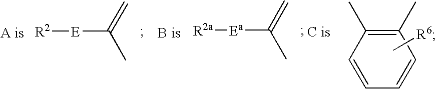

D is —N—, —NO—; E and E^{a} are each independently selected from a single bond, —O—, —S—, —SO—, and —SO₂; R² and R^{2a} are each independently selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C₅-C₂₀ aryl, and C₅-C₂₀ heteroaryl, wherein the aryl and heteroaryl groups are optionally substituted with one or more electron withdrawing groups including, but not limited to halogen, trihaloalkyl, cyano, nitro, carboxy or alkoxycarbonyl, or electron donating groups including, but not limited to hydroxyl, alkoxy, acyloxyl, amino, alkylamino, acylamino, mercapto, or alkylthio; and R⁶ is selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C 10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl and C1-C10 sulfonylalkyl.

In certain embodiments, A is

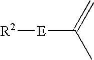

wherein R² is selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C1-C10 alkylsulfonylalkyl, C1-C10 alkylcarbonylaminoC1-C10 alkyl, C₃-C₂₀ heterocyclyl, C₃-C₂₀ heterocyclylalkyl, C5-C20 aralkyl, C5-C20 aryl, C5-C20 heteroaralkyl and C5-C20 heteroaryl, wherein the aryl and heteroaryl groups are optionally substituted with one or more electron withdrawing groups including, but not limited to halogen, trihaloalkyl, cyano, nitro, carboxy or alkoxycarbonyl, or electron donating groups including, but not limited to hydroxyl, alkoxy, acyloxyl, amino, alkylamino, acylamino, mercapto, or alkylthio.

In certain embodiments, B is

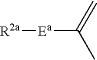

wherein R^{2a} is selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C1-C10 alkylsulfonylalkyl, C1-C10 alkylcarbonylaminoC1-C10alkyl, C₃-C₂₀ heterocyclyl, C₃-C₂₀ heterocyclylalkyl, C5-C20 aralkyl, C5-C20 aryl, C5-C20 heteroaralkyl and C5-C20 heteroaryl, wherein the aryl and heteroaryl groups are optionally substituted with one or more electron withdrawing groups including, but not limited to halogen, trihaloalkyl, cyano, nitro, carboxy or alkoxycarbonyl, or electron donating groups including, but not limited to hydroxyl, alkoxy, acyloxyl, amino, alkylamino, acylamino, mercapto, or alkylthio.

In certain embodiments, R² and R^{2a} are each independently selected from halogen, C1-C10 alkyl, C3-C10 cycloalkyl, C1-C10 hydroxyalkyl, C1-C10 alkoxycarbonylalkyl, C1-C10 alkylcarbonylaminoC1-C10alkyl, C₃-C₂₀ heterocyclyl, C₃-C₂₀ heterocyclylalkyl, C₅-C₂₀ aralkyl, C₅-C₂₀ aryl, C₅-C₂₀ heteroaralkyl and C₅-C₂₀ heteroaryl, where the aryl and heteroaryl groups are optionally substituted with one or more groups selected from alkyl, haloalkyl, halogen, alkoxycarbonyl, carboxy, hydroxy, alkylsulfonylamino, alkoxy and nitro.

In other embodiments, R² and R^{2a} are each independently selected from chloro, methyl, ethyl, n-butyl, tert-butyl, hydroxyethyl, methoxycarbonylethyl, methylcarbonylaminoethyl cyclopentyl, cyclohexyl, benzyl, phenyl, naphthyl, N-morpholinyl, 2-pyridinyl and 4-pyridinyl, where the phenyl and pyridinyl rings are optionally substituted with one or more groups selected from methyl, trifluoromethyl, chloro, methoxycarbonyl, carboxy, hydroxy, methylsulfonylamino, methoxy and nitro.

In other embodiments, R² and R^{2a} are each independently selected from chloro, methyl, ethyl, n-butyl, tert-butyl, hydroxyethyl, methoxycarbonylethyl, methylcarbonylaminoethyl cyclopentyl, cyclohexyl, N-morpholinyl, 4-pyridinyl, benzyl, phenyl, 2-pyridinyl, 4-nitrophenyl, 4-methoxyphenyl, p-tolyl, 4-trifluoromethyl, m-tolyl, o-tolyl, 3-methoxyphenyl, 2-methoxyphenyl, 2-chlorophenyl, naphthyl, 2-methoxycarbonylphenyl, 2-carboxyphenyl, 4-hydroxyphenyl, 4-methoxycarbonylphenyl, 4-aminophenyl, 4-carboxyphenyl and 4-methylsulfonylaminophenyl.

In other embodiments, R² is selected from chloro, methyl, ethyl, n-butyl, tert-butyl, hydroxyethyl, methoxycarbonylethyl, methylcarbonylaminoethyl cyclopentyl, cyclohexyl, N-morpholinyl, 4-pyridinyl, benzyl, phenyl, 2-pyridinyl, 4-nitrophenyl, 4-methoxyphenyl, p-tolyl, 4-trifluoromethyl, m-tolyl, o-tolyl, 3-methoxyphenyl, 2-methoxyphenyl, 2-chlorophenyl, naphthyl, 2-methoxycarbonylphenyl, 2-carboxyphenyl, 4-hydroxyphenyl, 4-methoxycarbonylphenyl, 4-aminophenyl, 4-carboxyphenyl and 4-methylsulfonylaminophenyl.

In other embodiments, R^{2a} is selected from chloro, methyl, ethyl, n-butyl, tert-butyl, hydroxyethyl, methoxycarbonylethyl, methylcarbonylaminoethyl cyclopentyl, cyclohexyl, N-morpholinyl, 4-pyridinyl, benzyl, phenyl, 2-pyridinyl, 4-nitrophenyl, 4-methoxyphenyl, p-tolyl, 4-trifluoromethyl, m-tolyl, o-tolyl, 3-methoxyphenyl, 2-methoxyphenyl, 2-chlorophenyl, naphthyl, 2-methoxycarbonylphenyl, 2-carboxyphenyl, 4-hydroxyphenyl, 4-methoxycarbonylphenyl, 4-aminophenyl, 4-carboxyphenyl and 4-methylsulfonylaminophenyl.

In certain embodiments, C is selected from

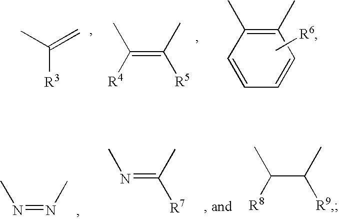

wherein R³ to R⁵ and R⁷ to R⁹ are each independently selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaninoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C1-C10 alkylsulfonylalkyl,C5-C20 aralkyl, C5-C20 aryl, C5-C20 heteroaralkyl and C5-C20 heteroaryl, wherein the aryl and heteroaryl groups are optionally substituted with one or more electron withdrawing groups including, but not limited to halogen, trihaloalkyl, cyano, nitro, carboxy or alkoxycarbonyl, or electron donating groups including, but not limited to hydroxyl, alkoxy, acyloxyl, amino, alkylamino, acylamino, mercapto, or alkylthio and R⁶ is selected from hydrogen; electron donating groups such as C1-C10 alkyl, C3-C10 cycloalkyl, hydroxyl, C1-C10 alkoxy, amino, C1-C10 acylamino, mercapto or C1-C10 alkylthio; and electron withdrawing groups such as C1-C10 acyl, halo, mono- or polyhaloalkyl, cyano, nitro, carboxy, C1-C10 alkoxycarbonyl, C1-C10 alkylsulfonyl, C5-C10 aryl, C1-C10 alkoxyalkyl, C1-C10 aminoalkyl, C1-C10 alkylaminoalkyl, C1-C10 hydroxyalkyl, C1-C10 carboxyalkyl, C1-C10 alkoxycarbonylalkyl, C1-C10 mercaptoalkyl, C1-C10 alkylthioalkyl, C1-C10 sulfonylalkyl, and C1-C10 alkylsulfonylalkyl.

In certain embodiments, C is

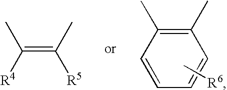

wherein R⁴ and R⁵ are each independently hydrogen, C1-C10 alkyl or C3-C10 cycloalkyl, and R⁶ is hydrogen or C1-C10 alkyl.

In certain embodiments, R⁴ and R⁵ are each independently selected from hydrogen and C1-C10 alkyl. In other embodiments, R⁴ and R⁵ are each independently hydrogen or methyl. In one embodiment, R⁴ is hydrogen or methyl. In other embodiment, R⁴ is methyl. In other embodiment R⁵ is hydrogen.

In certain embodiments, C is

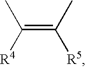

wherein R⁴ and R⁵ are each independently hydrogen or methyl.

In certain embodiments, C is

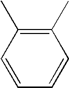

In certain embodiments, D is selected from —N—, —NO—, —NR¹⁰, —CR¹¹R¹²—, —CR¹³—, —S—, —SO—, and —SO₂—. In other embodiment, D is —N— or —NO—. In another embodiment, D is —N—.

In certain embodiments, E and E^{a} are each independently selected from a single bond, —CR¹⁴R¹⁵, —NR¹⁶, —O—, —S—, —SO—, and —SO₂—. In certain embodiments, E and E^{a} are each independently —O—, —S—, —SO—, or —SO₂—. In other embodiments, E is —SO₂—. In another embodiment, E^{a} is —SO₂—. In another embodiment E is a single bond. In another embodiment E^{a} is a single bond.

In certain embodiments, R⁶ is hydrogen.

In certain embodiments, the compounds provided herein are represented by structural formula:

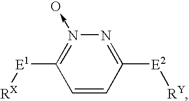

wherein E¹ and E² are each independently selected from a single bond, —O—, —S—, or —SO₂—; and R^{x} and R^{y} are each independently selected from halogen, C1-C10 alkyl, C3-C10 cycloalkyl, C1-C10 hydroxyalkyl, C1-C10 alkoxycarbonylalkyl, C1-C10 alkylcarbonylaminoC1-C10alkyl, C₃-C₂₀ heterocyclyl, C₃-C₂₀ heterocyclylalkyl, C₅-C₂₀ aralkyl, C₅-C₂₀ aryl, C₅-C₂₀ heteroaralkyl and C₅-C₂₀ heteroaryl, where the aryl and heteroaryl groups are optionally substituted with one or more groups selected from alkyl, haloalkyl, halogen, alkoxycarbonyl, carboxy, hydroxy, alkylsulfonylamino, alkoxy and nitro, with the proviso that when E¹ and E² are both SO₂, then at least one of R^{x} and R^{y} is not CH₃;.

In other embodiments, E¹ is chloro, —O— or —SO₂—. In other embodiment, E² is chloro, —O— or —SO₂—.

In certain embodiments, R^{x} and R^{y} are each independently selected from chloro, methyl, ethyl, n-butyl, tert-butyl, hydroxyethyl, methoxycarbonylethyl, methylcarbonylaminoethyl, cyclopentyl, cyclohexyl, benzyl, phenyl, naphthyl, N-morpholinyl, 2-pyridinyl and 4-pyridinyl, where the phenyl and pyridinyl rings are optionally substituted with one or more groups selected from methyl, trifluoromethyl, chloro, methoxycarbonyl, carboxy, hydroxy, methylsulfonylamino, methoxy and nitro.

In certain embodiments, R^{x} is phenyl, optionally substituted with one or more methyl, trifluoromethyl, chloro, methoxycarbonyl, carboxy, hydroxy, methylsulfonylamino, methoxy or nitro. In other embodiments, R^{y} is phenyl, optionally substituted with one or more methyl, trifluoromethyl, chloro, methoxycarbonyl, carboxy, hydroxy, methylsulfonylamino, methoxy or nitro.

In certain embodiments, the compounds have formula:

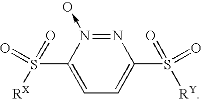

In other embodiments, the compounds have formula:

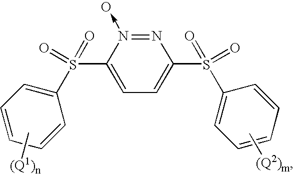

wherein m and n are integers selected from 0 to 4 and Q¹ and Q² are each independently selected from methyl, trifluoromethyl, chloro, methoxycarbonyl, carboxy, hydroxy, methylsulfonylamino, methoxy and nitro.

In certain embodiments, the compounds are selected from

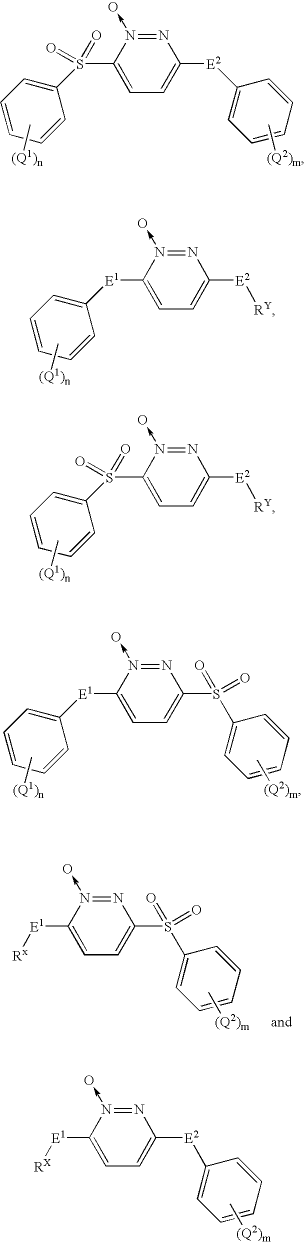

In other embodiments, the compounds have formula

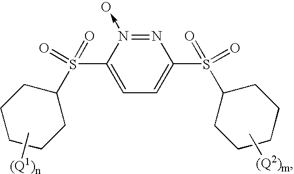

In certain embodiments, the compounds are selected from

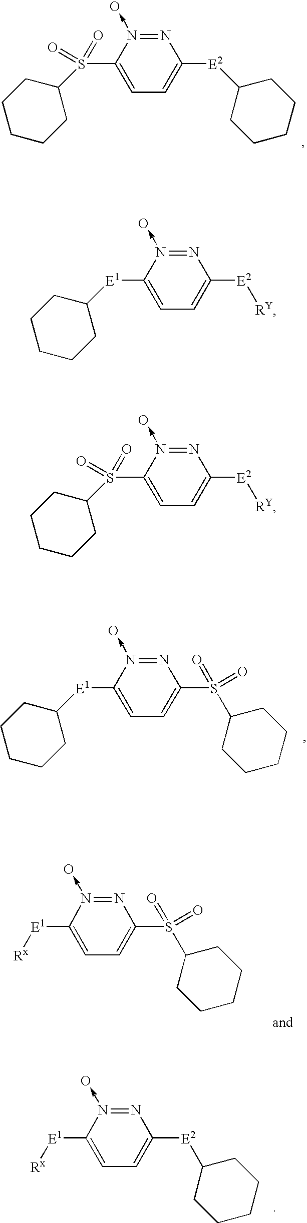

In certain embodiments, Q¹ and Q² are each independently selected from methyl, trifluoromethyl, chloro, methoxycarbonyl, carboxy, hydroxy, methylsulfonylamino, methoxy and nitro.

In certain embodiments m is 0 or 1. In other embodiments, n is 0 or 1.

In certain embodiments, the compounds are selected from

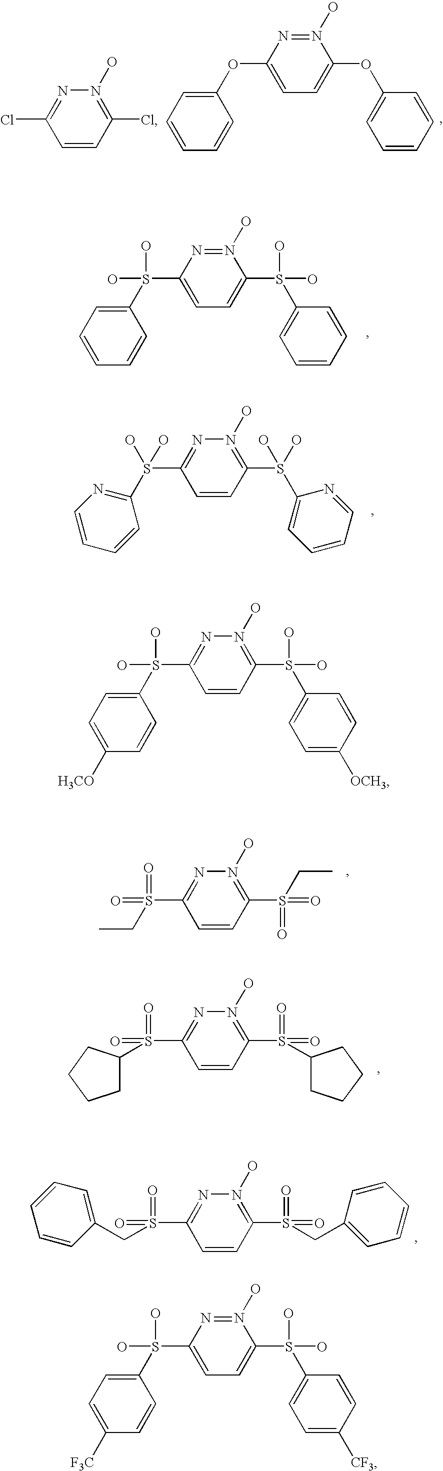

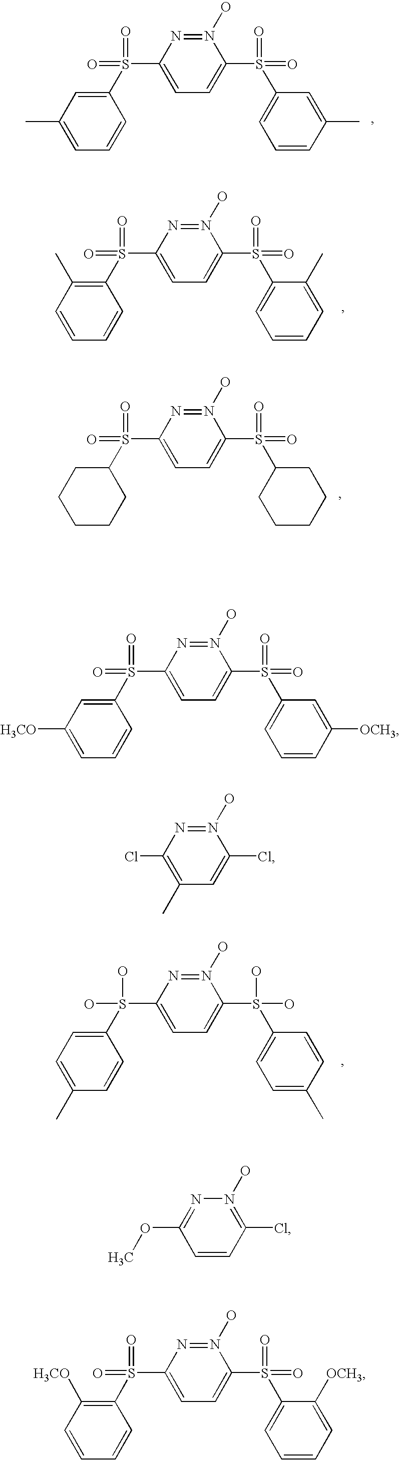

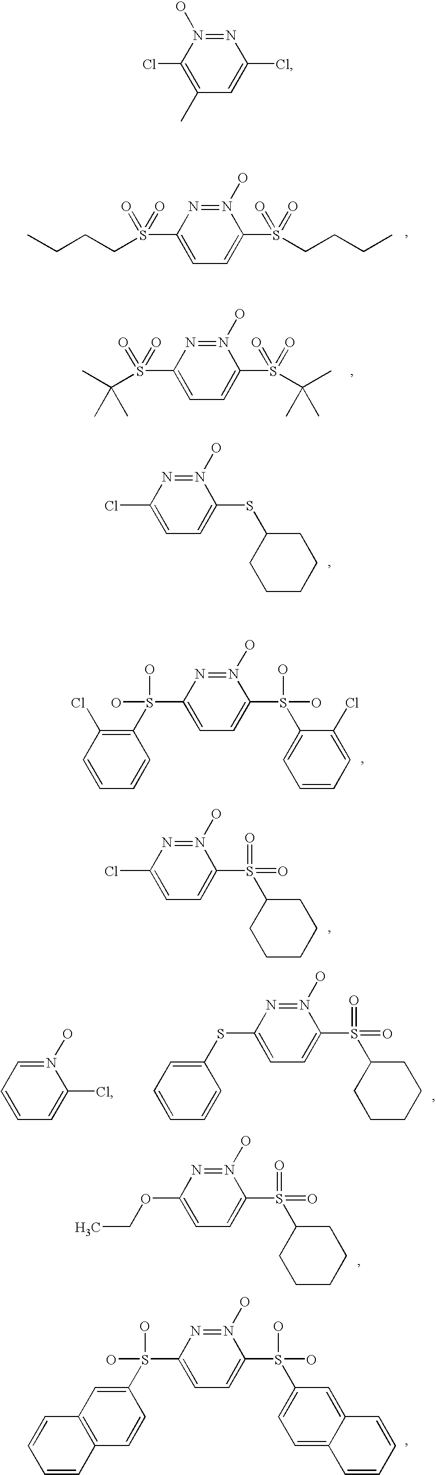

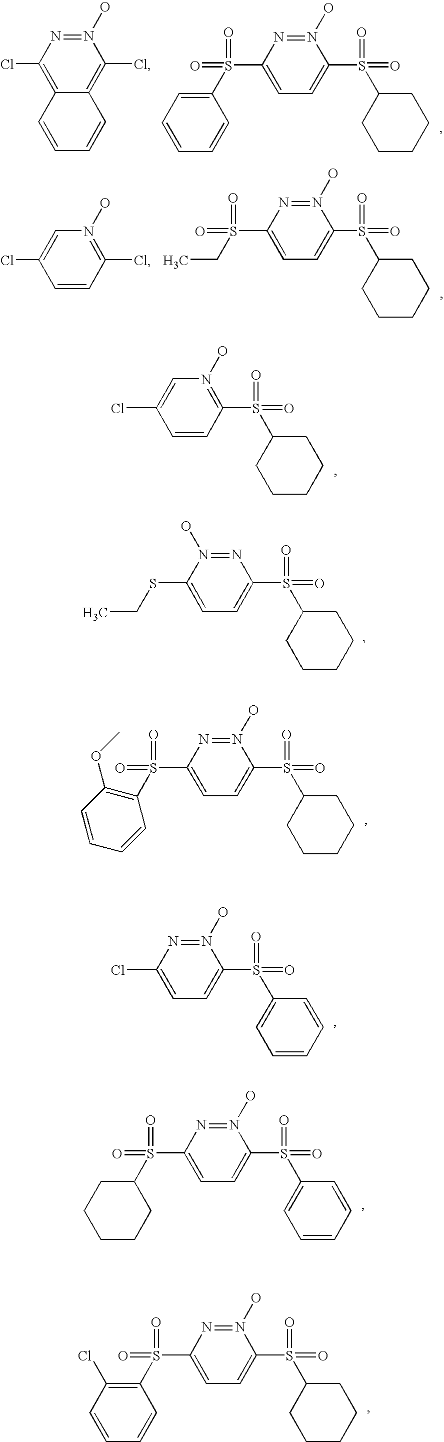

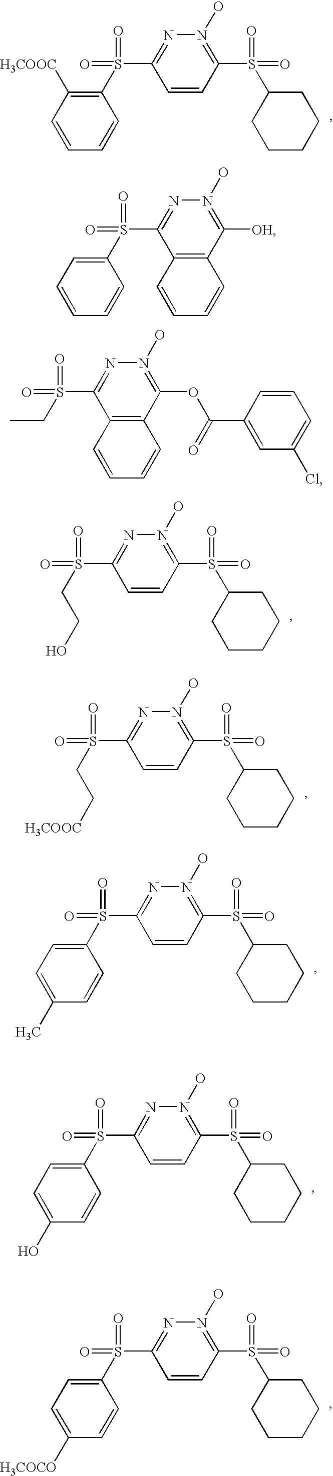

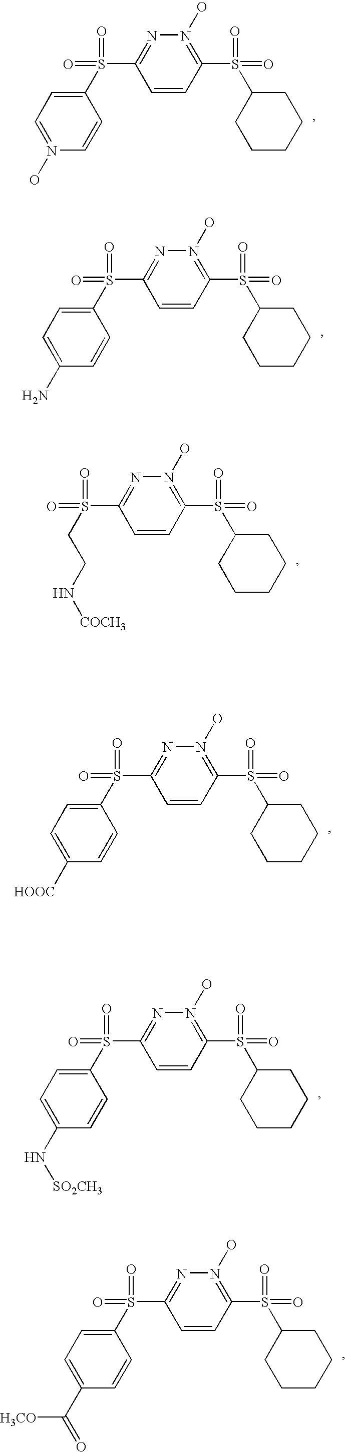

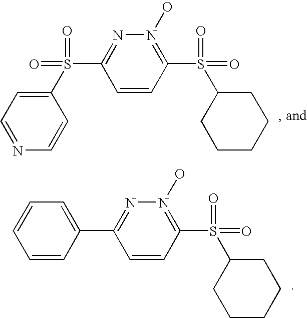

In certain embodiments the compounds for use in the compositions and methods provided herein are selected from Table 2. Table 2 provides in vitro data in HL60, human leukemic cells expressing high Bcl-2 and low Bcl-X_{L} levels, and A549 human lung cells for exemplary compounds, as described in Example 6. Average LC₅₀ is provided as follows: a=<50 μM, b=50-200 μM, c>200 μM and nc=data not calculated.

TABLE 2

| | | LC 50 (μM) | |
|---|---|---|---|
| S.NO. | Structure | A549 | HL-60 |
| 1 | 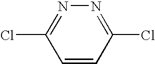 | c | c |
| 2 | 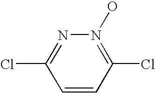 | c | a |
| 3 | 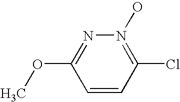 | c | c |
| 4 | 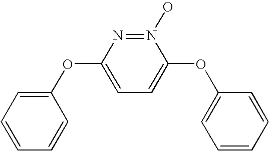 | c | c |
| 5 | 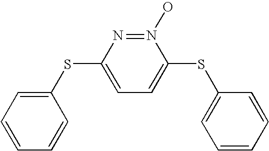 | n/c | c |
| 6 | 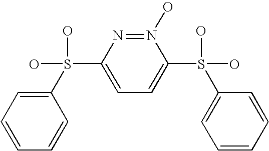 | c | a |
| 7 | 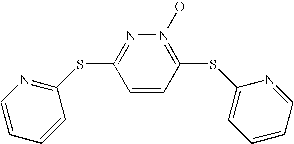 | c | c |
| 8 | 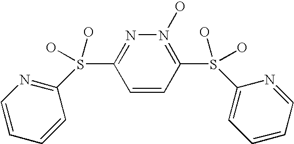 | c | b |
| 9 | 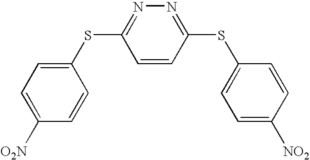 | c | c |
| 10 | 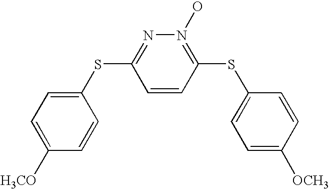 | c | c |
| 11 | 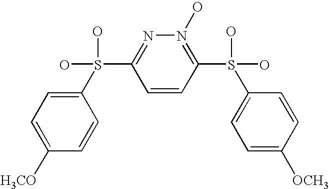 | c | b |
| 12 | 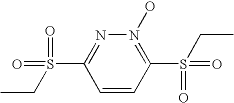 | b | b |
| 13 | 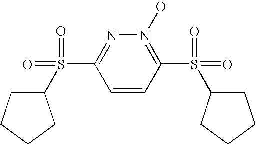 | c | a |
| 14 | 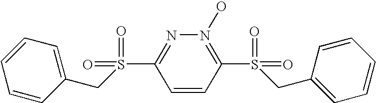 | c | c |
| 15 | 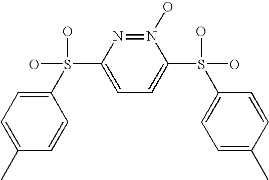 | c | c |
| 16 | 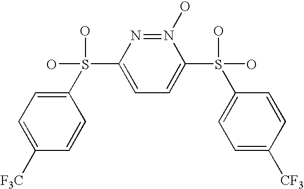 | c | c |
| 17 | 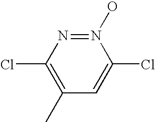 | c | c |
| 18 | 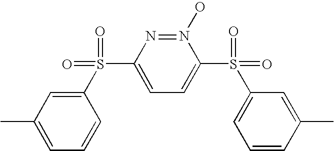 | c | c |
| 19 | 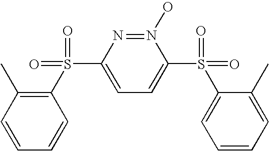 | c | b |
| 20 | 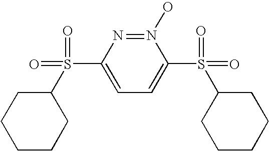 | c | a |
| 21 | 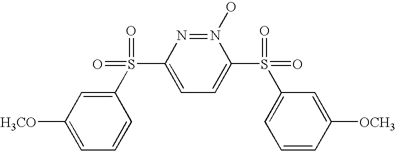 | c | c |
| 22 | 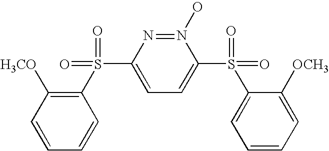 | c | a |
| 23 | 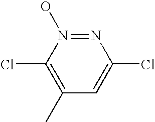 | c | c |
| 24 | 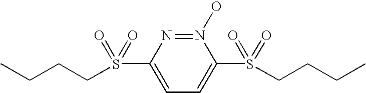 | c | b |
| 25 | 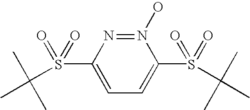 | c | b |
| 26 | 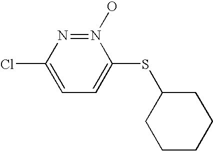 | c | c |
| 27 | 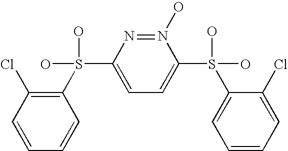 | c | a |
| 28 | 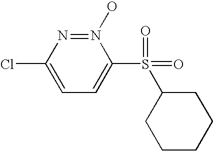 | c | a |
| 29 | 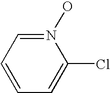 | c | c |
| 30 | 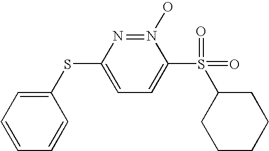 | c | c |
| 31 | 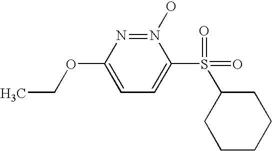 | c | c |
| 32 | 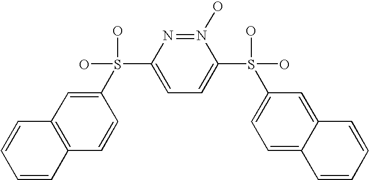 | c | c |
| 33 | 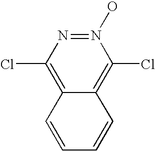 | b | b |
| 34 | 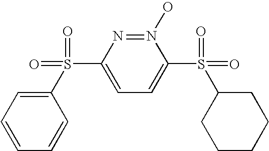 | c | a |
| 35 | 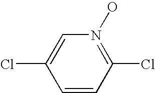 | c | c |
| 36 | 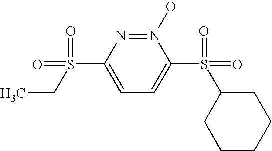 | b | a |
| 37 | 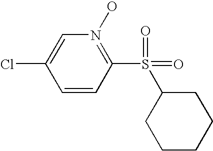 | c | c |
| 38 | 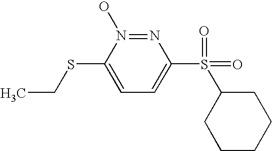 | c | c |
| 39 | 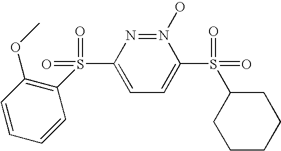 | b | a |
| 40 | 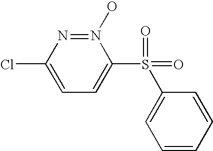 | c | a |
| 41 | 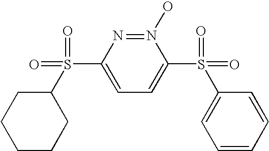 | c | a |
| 42 | 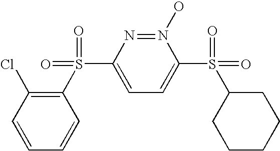 | b | a |
| 43 | 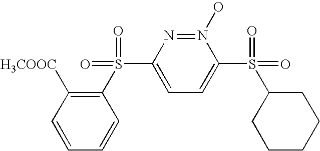 | c | a |
| 44 | 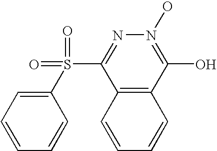 | c | c |
| 45 | 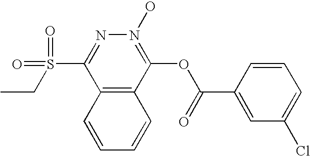 | c | c |
| 46 | 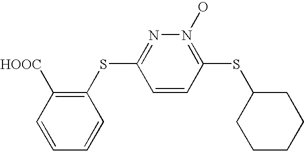 | c | c |
| 47 | 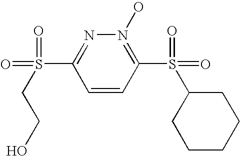 | c | c |
| 48 | 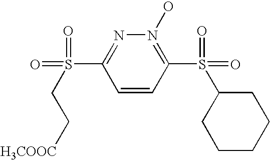 | c | a |
| 49 | 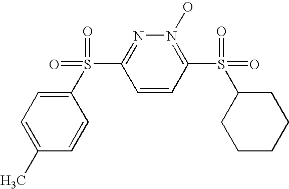 | c | b |
| 50 | 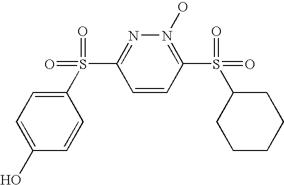 | c | b |
| 51 | 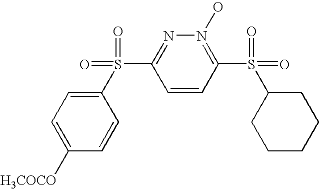 | c | b |
| 54 | 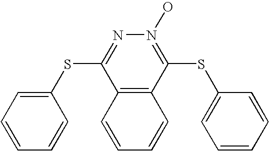 | c | c |
| 53 | 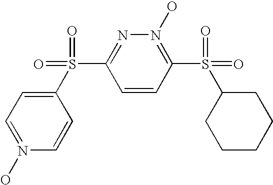 | c | b |
| 54 | 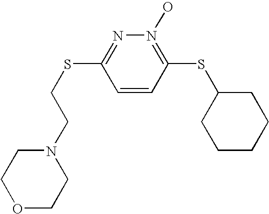 | c | c |
| 55 | 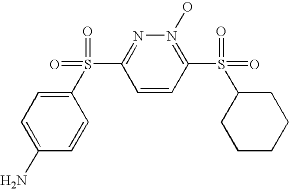 | c | a |
| 56 | 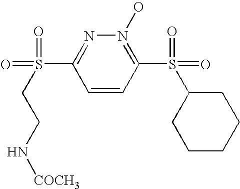 | c | c |
| 57 | 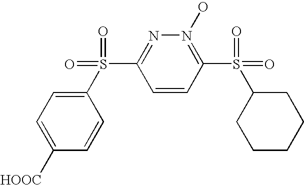 | c | c |
| 58 | 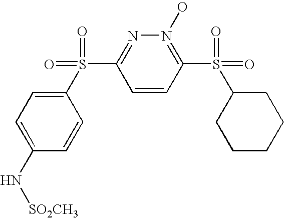 | c | c |
| 59 | 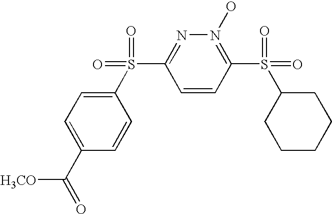 | c | a |
| 60 | 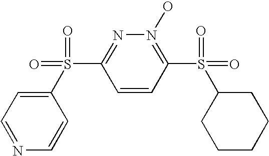 | c | b |
| 61 | 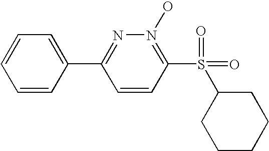 | c | c |

C. Preparation of the Compounds

The compounds belonging to Formula 8 can be prepared by standard synthetic methods known in the art, and are shown in Schemes 1-8. The examples that follow describe the exemplary embodiments and are not purported to limit the scope of the claimed subject matter. It is intended that the specification, together with the following examples, be considered exemplary only, with the scope and spirit of the claimed subject matter being indicated by the claims that follow these examples. Other embodiments within the scope of claims herein will be apparent to one skilled in the art from consideration of the specification described herein.

The bisthioether intermediate 12 can be prepared as shown in Scheme 1. The

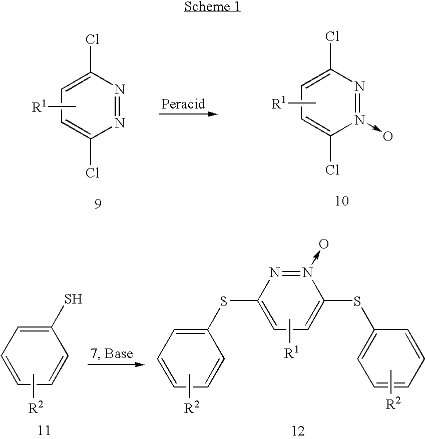

intermediates represented by 10 can be produced according to the procedures as described in the literature, which is incorporated herein by reference in its entirety (Tzung, S. P. et al., Nat. Cell Biol. 2001, 3, 183). Nucleophilic substitution reaction to produce 12 can be carried out in the presence of an inorganic base such as potassium carbonate, sodium carbonate, sodium hydroxide or potassium hydroxide or an organic base such as triethylamine, pyridine, piperidine, DBU or DBN. The solvent may, for example, be an aromatic hydrocarbon such as benzene or toluene; an ether such as diethyl ether, tetrahydrofuiran or dioxane; a halogenated hydrocarbon such as methylene chloride or chloroform or an aprotic polar solvent such as acetonitrile, dimethylformamide, pyridine or methyl ethyl ketone. The reaction temperature is usually from −50° to +150° C., preferably from 50° to 120° C. The reaction time is from 0.1 to 24 hours. The oxidation of the sulfur atoms in 12 leading to the corresponding sulfoxides and sulfones can be carried out by the use of oxidants such as m-chloroperoxybenzoic acid, perbenzoic acid, hydrogen peroxide, urea-hydrogen peroxide adduct, or Oxone. The reaction is carried out in a solvent which can be a halogenated hydrocarbon such as methylene chloride, chloroform, or carbon tetrachloride, an aromatic hydrocarbon such as benzene or toluene, an aprotic polar solvent such as acetonitrile, dimethylformamide, methyl ethyl ketone, tetrahydrofuiran or 1,4-dioxane. The reaction temperature is usually from −50° to +150° C., preferably from 20° to 110° C. The reaction time is from 2 to 24 hours. Alternatively, 12 can be prepared by nucleophilic substitution of 9 with 11 followed by oxidation.

The phthalazine intermediate 15 can be prepared as shown in Scheme 2. The reaction

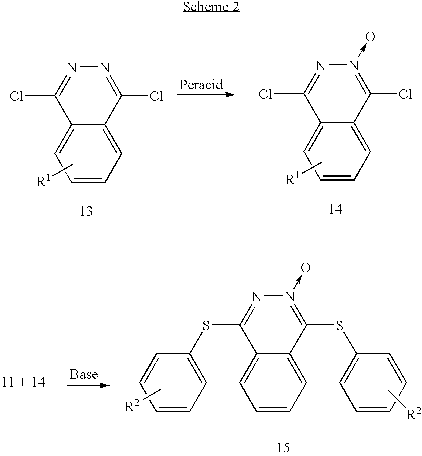

sequence is the same as those described for intermediate 12 in Scheme 1. Compound 15 can be further oxidized to the corresponding sulfoxide or sulfones with peracids.

The dioxide 19 can be prepared as shown in Scheme 3. The pyridazine derivatives 16 can be prepared by the procedure described in the literature, which is incorporated herein by reference in its entirety (Tzung, S. P. et al., Nat. Cell Biol. 2001, 3, 183). Halogenation of the methyl groups leading to 17 can be accomplished using the halogenation reagents such as N-bromosuccinimide or molecular bromine. The reaction is carried out in the presence or

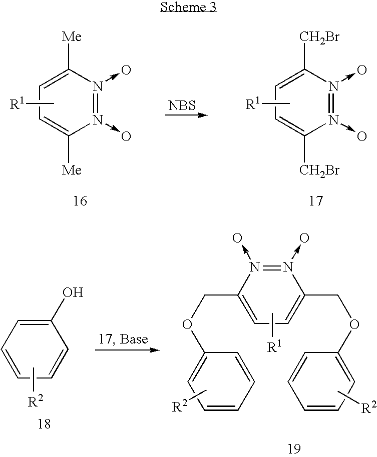

absence of catalysts such as benzoyl peroxide. The solvent for the reaction can be a halogenated hydrocarbon such as methylene chloride or chloroform, an aprotic polar solvent such as dimethylformamide or an acid such as acetic acid. The reaction temperature is usually from 0° to +150° C., preferably from 50° to 120° C. The reaction time is from 1 to 12 hours. Substitution reaction to produce compounds represented by 19 can be carried out in the presence of an inorganic base such as potassium carbonate, sodium carbonate, sodium hydroxide or potassium hydroxide or an organic base such as triethylamine, piperidine, pyridine, DBU or DBN. The solvent may be an aromatic hydrocarbon such as benzene or toluene; an ether such as diethyl ether, tetrahydrofuran or dioxane; or an aprotic polar solvent such as acetonitrile, dimethylformamide, dimethylsulfoxide or methyl ethyl ketone. The reaction temperature is usually from 20° to +150° C., preferably from 50° to 120° C. The reaction time is from 1 to 24 hours.

The amide and ester derivatives 22a and 22b respectively can be prepared as outlined in Scheme 4. Oxidation reaction leading to the formation carboxylic acid derivatives represented by the formula 20 can be carried out in the presence of oxidants such as molecular oxygen, air, or hydrogen peroxide in a solvent such as water, tetrahydrofuran, dioxane, ethanol, methanol, acetic acid or a combination of those. The presence of a catalyst such as benzoyl peroxide, manganese acetate or cobalt acetate in addition to salts such as sodium bromide or potassium bromide may be desirable. The reaction temperature may range from 0° to +150° C., preferably from 50° to 120° C. The reaction time is from 1 to 24 hours. Formation of derivatives represented by the formula 22a and 22b can be

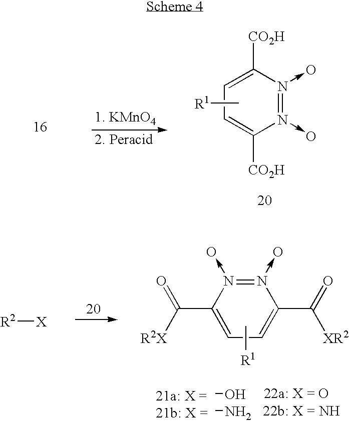

accomplished by the reaction of nucleophiles with the intermediates such as the acid chlorides, bromides or mixed anhydrides derived from 20.

Ether derivatives such as 24 can be prepared by displacement reaction of an alkyl halide with a phenoxide derivative of 23 as shown in Scheme 5. The formation of the aryl ether is

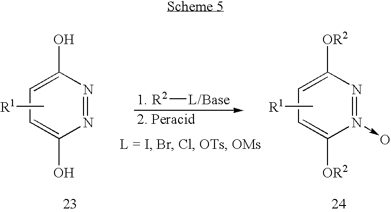

carried out in the presence of a base such as potassium carbonate, sodium carbonate, sodium hydroxide or potassium hydroxide. The reaction is carried out in polar, aprotic solvents such as dimethylsulfoxide, dimethyl formamide, methyl ethyl ketone, or dioxane at a temperature from 0° to +150° C., preferably from 50° to 120° C. The reaction time is from 1 to 24 hours. The N-oxidation reaction leading to 24 can be carried out using oxidizing agents such as hydrogen peroxide or m-chloroperoxybenzoic acid according to the previously described procedures.

Preparation of 3-6 diarylpyridazine derivatives 26 can be accomplished by coupling reaction between 9 and compounds represented by the formula 25 (Scheme 6) under Suzuki conditions where boronic acid derivative is treated with the halide in the presence of a base such as triphenylphosphine, tri-t-butylphosphine, caesium carbonate, potassium carbonate, sodium acetate, or triethyl amine in the presence of transition metal catalyst such as

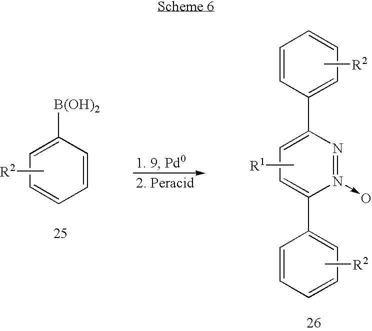

palladium acetate. The solvent for the reaction may be an aromatic hydrocarbon such as benzene or toluene; an ether such as tetrahydrofuran, dioxane, 1,2 dimethoxy ethane, or bis-2-methoxyethyl ether; a halogenated hydrocarbon such as methylene chloride, chloroform, 1,2-dichloroethane; an aprotic polar solvent such as acetonitrile or dimethylformamide or a protic polar solvent such as ethanol, methanol or water. The reaction temperature is usually from 0° to +150° C., preferably from 50° to 120° C. The reaction time is from 1 to 24 hours. The N-oxidation reaction leading to 26 can be carried out using oxidizing agents such as hydrogen peroxide or m-chloroperoxybenzoic acid according to the previously described procedures.

Coupling reaction between 9 and compounds represented by the formula 27a,b to generate stilbene type compounds represented by the formula 28 (Scheme 7) can be carried

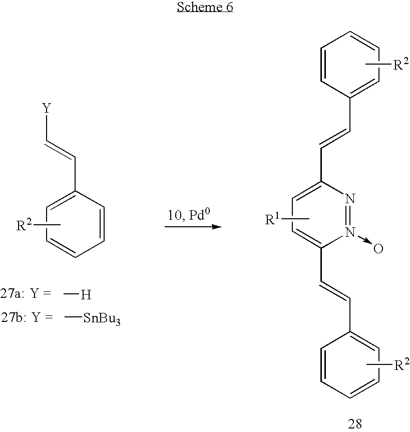

out under Stille conditions where the organometallic tin derivatives such as 27b is treated with the halide in the presence of a base such as triphenylphosphine, potassium carbonate, sodium acetate, or triethyl amine and in the presence of transition metal catalyst such as palladium acetate or chloride. The solvent for the reaction may be an aromatic hydrocarbon such as 2-methoxyethyl ether; a halogenated hydrocarbon such as methylene chloride, chloroform, 1,2-dichloroethane; an aprotic polar solvent such as acetonitrile or dimethylformamide. The reaction temperature is usually from 0° to +150° C., preferably from 50° to 120° C. Formation of 28 can also be accomplished by transition metal catalyzed coupling of 9 with an olefin such as 27a under Heck conditions, which involves the palladium catalyzed substitution of a vinylic hydrogen with an aryl halide such as 9. The reaction can be carried out in the presence of a catalyst such as palladium acetate and is promoted by a base such as potassium carbonate, sodium acetate, or triethyl amine.

Coupling reaction between 9 and alkynes 29 to generate the acetylenic compounds represented by the formula 30 (Scheme 8) can be carried out in the presence of a base such as

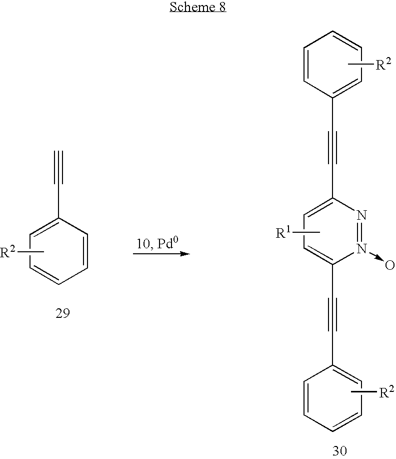

triphenylphosphine, potassium carbonate, sodium acetate, or triethyl amine and in the presence of transition metal catalyst such as palladium acetate or chloride. The reaction is facilitated by the presence of salts such as copper (I) iodide. The solvent for the reaction may be an aromatic hydrocarbon such as benzene or toluene; an ether such as tetrahydrofliran, dioxane, 1,2 dimethoxy ethane, or bis-2-methoxyethyl ether; a halogenated hydrocarbon such as methylene chloride, chloroform, 1,2-dichloroethane; an aprotic polar solvent such as acetonitrile or dimethylformamide. The reaction temperature is usually from 0° to +150° C., preferably from 50° to 120° C. The reaction time is from 1 to 24 hours.

D. Pharmaceutical Compositions

The compounds provided herein can be used as such, be administered in the form of pharmaceutically acceptable salts derived from inorganic or organic acids, or used in combination with one or more pharmaceutically acceptable excipients. The phrase “pharmaceutically acceptable salt” means those salts which are, within the scope of sound medical judgment, suitable for use in contact with the tissues without undue toxicity, irritation, allergic response, and the like, and are commensurate with a reasonable benefit/risk ratio. Pharmaceutically acceptable salts are well known in the art. The salts can be prepared either in situ during the final isolation and purification of the compounds provided herein or separately by reacting the acidic or basic drug substance with a suitable base or acid respectively. Typical salts derived from organic or inorganic acids salts include, but are not limited to hydrochloride, hydrobromide, hydroiodide, acetate, adipate, alginate, citrate, aspartate, benzoate, bisulfate, gluconate, fumarate, hydroiodide, lactate, maleate, oxalate, palmitoate, pectinate, succinate, tartrate, phosphate, glutamate, and bicarbonate. Typical salts derived from organic or inorganic bases include, but are not limited to lithium, sodium, potassium, calcium, magnesium, ammonium, monoalkylammonium such as meglumine, dialkylammonium, trialkylammonium, and tetralkylammonium.

The mode of administration of the pharmaceutical compositions can be oral, rectal, intravenous, intramuscular, intracisternal, intravaginal, intraperitoneal, bucal, subcutaneous, intrasternal, nasal, or topical. The compositions can also be delivered at the target site through a catheter, an intracoronary stent (a tubular device composed of a fine wire mesh), a biodegradable polymer, or biological carriers including, but are not limited to antibodies, biotin-avidin complexes, and the like. Dosage forms for topical administration of a compound provided herein include powders, sprays, ointments and inhalants. The active compound is mixed under sterile conditions with a pharmaceutically acceptable carrier and any needed preservatives, buffers or propellants. Opthalmic formulations, eye ointments, powders and solutions are also provided herein.

Actual dosage levels of active ingredients and the mode of administration of the pharmaceutical compositions provided herein can be varied in order to achieve the effective therapeutic response for a particular patient. The phrase “therapeutically effective amount” of the compound provided herein means a sufficient amount of the compound to treat disorders, at a reasonable benefit/risk ratio applicable to any medical treatment. It will be understood, however, that the total daily usage of the compounds and compositions of the provided will be decided by the attending physician within the scope of sound medical judgment. The total daily dose of the compounds provided herein may range from about 0.0001 to about 1000 mg/kg/day. For purposes of oral administration, doses can be in the range from about 0.001 to about 5 mg/kg/day. If desired, the effective daily dose can be divided into multiple doses for purposes of administration; consequently, single dose compositions may contain such amounts or submultiples thereof to make up the daily dose. The specific therapeutically effective dose level for any particular patient will depend upon a variety of factors including the disorder being treated and the severity of the disorder; medical history of the patient, activity of the specific compound employed; the specific composition employed, age, body weight, general health, sex and diet of the patient, the time of administration, route of administration, the duration of the treatment, rate of excretion of the specific compound employed, drugs used in combination or coincidental with the specific compound employed; and the like.

The compounds provided can be formulated together with one or more non-toxic pharmaceutically acceptable diluents, carriers, adjuvants, and antibacterial and antifungal agents such as parabens, chlorobutanol, phenol, sorbic acid, and the like. Proper fluidity can be maintained, for example, by the use of coating materials such as lecithin, by the maintenance of the required particle size in the case of dispersions, and by the use of surfactants. In some cases, in order to prolong the effect of the drug, it is desirable to decrease the rate of absorption of the drug from subcutaneous or intramuscular injection. This can be accomplished by suspending crystalline or amorphous drug substance in a vehicle having poor water solubility such as oils. The rate of absorption of the drug then depends upon its rate of dissolution, which, in turn, may depend upon crystal size and crystalline form. Prolonged absorption of an injectable pharmaceutical form can be achieved by the use of absorption delaying agents such as aluminum monostearate or gelatin.

The compound provided herein can be administered enterally or parenterally in solid or liquid forms. Compositions suitable for parenteral injection may comprise physiologically acceptable, isotonic sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), vegetable oils (such as olive oil), injectable organic esters such as ethyl oleate, and suitable mixtures thereof. These compositions can also contain adjuvants such as preserving, wetting, emulsifying, and dispensing agents. Suspensions, in addition to the active compounds, may contain suspending agents such as ethoxylated isostearyl alcohols, polyoxyethylene sorbitol and sorbitan esters, microcrystalline cellulose, aluminum metahydroxide, bentonite, agar-agar and tragacanth, or mixtures of these substances.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or (a) fillers or extenders such as starches, lactose, sucrose, glucose, mannitol and silicic acid; (b) binders such as carboxymethylcellulose, alginates, gelatin, polyvinylpyrrolidone, sucrose and acacia; (c) humectants such as glycerol; (d) disintegrating agents such as agar-agar, calcium carbonate, potato or tapioca starch, alginic acid, certain silicates and sodium carbonate; (e) solution retarding agents such as paraffin; (f) absorption accelerators such as quaternary ammonium compounds; (g) wetting agents such as cetyl alcohol and glycerol monostearate; (h) absorbents such as kaolin and bentonite clay and (i) lubricants such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavoring and perfuming agents.

Compositions for rectal or vaginal administration are preferably suppositories which can be prepared by mixing the compounds provided herein with suitable non-irritating excipients or carriers such as cocoa butter, polyethylene glycol or a suppository wax which are solid at room temperature but liquid at body temperature and therefore melt in the rectum or vaginal cavity and release the active compound.

Compounds provided herein can also be administered in the form of liposomes. Methods to form liposomes are known in the art (Prescott, Ed., Methods in Cell Biology 1976, Volume XIV, Academic Press, New York, N.Y.) As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound provided herein, stabilizers, preservatives, excipients and the like. The preferred lipids are natural and synthetic phospholipids and phosphatidyl cholines (lecithins).

The compounds provided herein can also be administered in the form of a ‘prodrug’ wherein the active pharmaceutical ingredients, represented by Formulas 1-3, are released in vivo upon contact with hydrolytic enzymes such as esterases and phophatases in the body. The term “pharmaceutically acceptable prodrugs” as used herein represents those prodrugs of the compounds provided herein, which are, within the scope of sound medical judgment, suitable for use in contact with the tissues without undue toxicity, irritation, allergic response, and the like, commensurate with a reasonable benefit/risk ratio, and effective for their intended use. A thorough discussion is provided in Higuchi, T. and Stella, V., Pro-drugs as Novel Delivery Systems, V. 14 of the A.C.S. Symposium Series; Edward B. Roche, Ed., Bioreversible Carriers in Drug Design 1987, American Pharmaceutical Association and Pergamon Press), which is incorporated herein by reference.

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The compounds or pharmaceutically acceptable derivatives may be packaged as articles of manufacture containing packaging material, a compound or pharmaceutically acceptable derivative thereof provided herein, which is effective for modulating the activity of a Bcl-2 protein, or for treatment, prevention or amelioration of one or more symptoms of Bcl-2 protein-mediated diseases or disorders, or diseases or disorders in which Bcl-2 protein-mediated activity, is implicated, within the packaging material, and a label that indicates that the compound or composition, or pharmaceutically acceptable derivative thereof, is used for modulating the activity of a Bcl-2 protein, or for treatment, prevention or amelioration of one or more symptoms of Bcl-2 protein-mediated diseases or disorders, or diseases or disorders in which Bcl-2 protein activity is implicated.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder in which a Bcl-2 protein is implicated as a mediator or contributor to the symptoms or cause.

E. Evaluation of the Activity of the Compounds

The biological activity of the compounds provided herein were assessed in cellular system by the method similar to the one described by Amundson et al. (Amundson, S. A. et al, Cancer Research 2000, 60, 6101-6110), which is incorporated herein by reference in its entirety. The compounds were tested for acute cytotoxicity (apoptotic activity) using two human cancer cell lines expressing high levels of either Bcl-2 or BCl-X_{L}. HL60, human leukemic cells expressing high Bcl-2 and low BCl-X_{L} levels, and A549 human lung cells expressing low Bcl-2 and high BCl-X_{L} levels, were incubated with the compounds at concentrations between 0.5 and 1000 μg/mL for 4 hours at 37° C., 5% CO₂. The number of viable cells were measured by mitochondrial transformation of Alamar Blue to a fluorescent dye. LC₅₀ values for each test compound and controls are provided in the individual examples that follow.

F. Combination Therapy

The compounds provided herein may be administered as the sole active ingredient or in combination with other active ingredients. Other active ingredients that may be used in combination with the compounds provided herein include known Bcl-2 protein antagonists, other compounds for use in treating, preventing, or ameliorating one or more symptoms of Bcl-2 protein mediated diseases and disorders, anti-angiogenesis agents, anti-tumor agents, and other cancer treatments. Such compounds include, in general, but are not limited to, alkylating agents, toxins, antiproliferative agents and tubulin binding agents. Classes of cytotoxic agents for use herein include, for example, the anthracycline family of drugs, the vinca drugs, the mitomycins, the bleomycins, the cytotoxic nucleosides, the pteridine family of drugs, diynenes, the maytansinoids, the epothilones, the taxanes and the podophyllotoxins.

G. Methods of Use of the Compounds and Compositions

The compounds and compositions provided herein are useful in methods of treatment, prevention, or amelioration of one or more symptoms of Bcl-2 protein-mediated diseases or disorders, including but not limited to Bcl-2 and Bcl-XL mediated diseases or disorders. In certain embodiments, the diseases are characterized by overexpression of to Bcl-2 and Bcl-XL protein. In certain embodiments the diseases or disorders include, but are not limited to, cancers, tumors, hyperproliferative diseases, acquired immune deficiency syndrome, degenerative conditions, and vascular diseases. In certain embodiments, the cancers include, but are not limited to B-cell lymphoma including B-cell lymphoma-2, B-cell leukemia, skin cancer, pancreatic cancer, ovarian cancer, liver cancer, bladder cancer, adrenal carcinoma, breast cancer, prostate cancer, colorectal cancer including colorectal adenocarcinomas, follicular lymphoma.

Methods of modulating the activity of a Bcl-2 protein, including but not limited to an anti-apoptotic Bcl-2 protein, Bcl-2 and BCl-X_{L}, by administering one or more of the compounds or compositions provided herein are also provided.

Methods of antagonizing a Bcl-2 protein, including but not limited to an anti-apoptotic Bcl-2 protein, including Bcl-2 and BCl-X_{L}, by contacting a composition containing the Bcl-2 protein with one or more of the compounds or compositions provided herein are also provided.

Methods of altering the interaction of an anti-apoptotic Bcl-2 protein, including but not limited to Bcl-2 and BCl-X_{L}, and a pro-apoptotic Bcl-2 protein, including but not limited to Bax, Bak, Bid and Bad, by contacting a composition containing the anti-apoptotic Bcl-2 protein and the pro-apoptotic Bcl-2 protein with a compound or composition provided herein, are also provided.

Methods of inducing apoptosis by administering one or more of the compounds or compositions provided herein are also provided.

The following examples are exemplary only and are not intended to limit the scope of the subject matter claimed herein.

### EXAMPLE 1

### Preparation of 3,6-bis(benzenesulfonyl)pyridazine-1-oxide

Step 1. A mixture of 3,6-dichloropyridazine (1.0 g, 6.7 mmol) and urea-hydrogen peroxide adduct (0.6 g, 6.4 mmol) in methylene chloride (25 ml) was stirred at ambient temperature. Trifluoroacetic anhydride (0.85 ml) dissolved in methylene chloride (2 ml) was slowly added and the solution was heated at reflux for 2 hours. A second batch of the urea-hydrogen peroxide adduct (0.2 g, 2.13 mmol) was added followed by the addition of trrfluoroacetic anhydride (0.3 ml) in methylene chloride (2 ml). Solution was refluxed for 4 hr and solvent evaporated. Residue was chromatographed on silica gel (hexane:ethyl acetate, 8:2) to furnish pyridazine, 3,6-dichloro-, 1-oxide (0.7 g, 4.2 mmol). LC-MS (API-ES, pos.) M⁺ 164.9, 166.9; ¹H NMR (DMSO-d6) 7.58 (1H, d, J=8.4 Hz), 8.37 (1H, d, J=8.4 Hz) ppm.

Step 2. A mixture of pyridazine, 3,6-dichloro-, 1-oxide (0.25 g, 1.5 mmol), thiophenol (0.33 g, 3.0 mmol), and potassium carbonate (0.42 g, 3.0 mmol) in dimethylsulfoxide (5 ml) was stirred at 110° C. for 2 hours. Water was added and product extracted with ethyl acetate. Evaporation of the solvent in vacuo furnished a residue which was subjected to flash chromatography on silica gel (hexane:ethyl acetate, 3:1) to afford pyridazine, 3,6-bis(phenylthio)-, 1-oxide (0.14 g, 0.45 mmol). LC-MS (API-ES, pos.) M+H⁺ 313.0; ¹H NMR (CDCl₃) 6.41 (1H, d, J=8.7 Hz), 6.63 (1H, d, J=8.7 Hz), 7.38-7.60 (10H, m) ppm.

The LC₅₀ value for this compound in HL-60 cell lines was 58.9 μM.

Step 3. Pyridazine, 3,6-bis(phenylthio)-, 1-oxide (0.14 g, 0.45 mmol) was dissolved in methylene chloride (10 ml) and m-chloroperoxybenzoic acid (0.56 g, 70%, 2.27 mmol) was added. Solution was stirred at ambient temperature over night. The solution was washed with saturated aqueous sodium bicarbonate solution followed by evaporation in vacuo. The residue was subjected to flash chromatography on silica gel (hexane:ethyl acetate, 7:3-6:4) to afford pyridazine, 3,6-bis(phenylsulfonyl)-, 1-oxide (0.05 g, 0.15 mmol). LC-MS (API-ES, pos.) M+H⁺ 377.0; ¹H NMR (DMSO-d6) 7.64-7.75 (4H, m), 7.78-7.87 (2H, m), 7.98-8.05 (4H, m), 8.16 (1H, d, J=8.4 Hz), 8.90 (1H, d, J=8.4 Hz) ppm.

The LC₅₀ value for this compound in A549 and HL-60 cell lines were 268.32 μM and 20.46 μM respectively.

### EXAMPLE 2

### Preparation of 3,6-diphenoxvpyridazine-1-oxide

A mixture of pyridazine, 3,6-dichloro-, 1-oxide (0.10 g, 0.61 mmol), phenol (0.12 g, 1.3 mmol), and potassium carbonate (0.17 g, 1.2 mmol) in dimethylsulfoxide (5 ml) was stirred at 110° C. for 2 hours. Water was added and product extracted with ethyl acetate. Evaporation of the solvent in vacuo furnished a residue which was subjected to flash chromatography on silica gel (hexane:ethyl acetate, 3:1) to afford pyridazine, 3,6-diphenoxy-, 1-oxide (0.09 g, 0.32 mmol). LC-MS (API-ES, pos.) M+H⁺ 281.1; ¹H NMR (DMSO-d6) 6.98-7.04 (2H, m), 7.14 (1H, d, J=9.0 Hz), 7.17 (1H, m), 7.27-7.35 (3H, m), 7.36-7.43 (2H, m), 7.46-7.53 (2H, m), 7.98 (1H, d, J=9.0 Hz) ppm.

The LC₅₀ value for this compound in A549 and HL-60 cell lines were both >500 μM.

### EXAMPLE 3

### Preparation of 3,6-bis-cyclohexanesulfonyl-pyridazine-1-oxide

Step 1. A suspension of sodium hydride (40 mmol) in anhydrous dimethylformamide (100 ml) was stirred at −20° C. Cyclohexylmercaptan (4.44 g, 38.2 mmol) dissolved in anhydrous dimethylformamide (50 ml) was slowly added and the solution stirred for 15 min. Pyridazine-3,6-dichloro-1-oxide (3.0 g, 18.2 mmol) dissolved in anhydrous dimethylformamide (50 ml) was slowly added and the solution was stirred at ambient temperature for 2 h. Solution was then added to ice-water and the precipitated solid separated by filtration followed by vacuum drying. The residue was subjected to flash chromatography on silica gel (eluent, hexane:ethyl acetate, 80:20, 60:40) to afford 3,6-bis-cyclohexylsulfanyl-pyridazine 1-oxide (4.95 g, 15.3 mmol). LC-MS (API-ES, pos.) M+H⁺ 325.1.

Step 2. 3,6-Bis-cyclohexylsulfanyl-pyridazine 1-oxide (4.9 g, 15.1 mmol).was dissolved in methylene chloride (200 ml) and m-chloroperoxybenzoic acid (18.6 g, 70%, 75.5 mmol) was added. Solution was stirred at ambient temperature over night. The solution was washed with saturated aqueous sodium bicarbonate solution followed by evaporation in vacuo. The residue was subjected to flash chromatography on silica gel (hexane:ethyl acetate, 70:30, 60:40) to afford 3,6-bis-cyclohexanesulfonyl-pyridazine-1-oxide (2.2 g, 5.7 rumol). LC-MS (API-ES, pos.) M+H⁺ 389.1; ¹H NMR (CDCl₃) 8.54 (1H, d, J=8.2 Hz), 7.84 (1H, d, J=8.2 Hz), 4.01 (1H, m), 3.59 (1H, m), 1.15-2.1 (20H, m) ppm.

The LC₅₀ value for this compound in A549 and HL-60 cell lines were 191.5 μM and 32.3 μM respectively.

### EXAMPLE 4

### Preparation of 3-chloro-6-cyclohexanesulfonyl-pyridazine-1-oxide

Step 1. To a stirred solution of cyclohexyl mercaptan (2.54 g, 21.8 mmol) and sodium hydroxide (0.87 g, 21.8 mmol) in water (23 ml) at 0° C. was slowly added a solution of 3,6-dichloropyridazine-1-oxide (3.0 g, 18.2 mmol) in dioxane (6 ml). The mixture was stirred for 1 hr with a mechanical stirrer at this temperature. Water was added and the precipitate was separated by filtration. The residue was dissolved in a small amount of methylene chloride and allowed to stand at ambient temperature. When some crystals appeared, a small amount of ether was added. Upon stirring, white crystalline material appeared which was separated by filtration to furnish 3-chloro-6-cyclohexylsulfanyl-pyridazine 1-oxide (2.68 g, 10.9 mmol). LC-MS (API-ES, pos.) M+H⁺ 245.0, M+Na⁺ 267.0; ¹H NMR (CDCl₃) 7.48 (1H, d, J=9.0 Hz), 7.09 (1H, d, J=9.0 Hz), 3.25-3.36 (1H, m), 1.97-2.08 (2H, m), 1.78-1.90 (2H, m), 1.30-1.72 (6H, m) ppm.

Step 2. A mixture of 3-chloro-6-cyclohexylsulfanyl-pyridazine 1-oxide (3.4 g, 13.89 mmol) and mcpba (10.27 g, 41.67 mmol, 70%) in methylene chloride (150 mL) was stirred at ambient temperature over night. The solution was washed with saturated aqueous sodium bicarbonate solution and evaporated to afford a residue which was dissolved in methylene chloride and passed through a plug of silica gel to afford 3-chloro-6-cyclohexanesulfonyl-pyridazine-1-oxide (2.5 g, 9.03 mmol). LC-MS (API-ES, pos.) M+H⁺ 276.9, M+Na⁺ 298.9; ¹H NMR (CDCl₃) 8.28 (1H, d, J=8.4 Hz), 7.29 (1H, d, J=8.4 Hz), 3.95-4.05 (1H, m), 1.15-2.0 (10H, m) ppm.

The LC₅₀ value for this compound in A549 and HL-60 cell lines were 188.45 μM and 20.16 μM respectively.

### EXAMPLE 5

### Preparation of 3-benzenesulfonyl-6-cyclohexanesulfonyl-pyridazine-1-oxide

Step 1. A mixture of 3-chloro-6-cyclohexylsulfanyl-pyridazine 1-oxide (0.50 g, 2.04 mmol), thiophenol (0.23 g, 2.04 mmol), and potassium carbonate (0.34 g, 2.46 mmol) in DMSO (10 mL) was stirred at 1100° C. for 1 hr under nitrogen. Water was added and product extracted with ethyl acetate. Organic layer was washed with water, dried (sodium sulfate) and solvent removed. The residue was crystallized from mehtylene chloride-ether to furnish 6-cyclohexylsulfanyl-3-phenylsulfanyl-pyridazine-1-oxide (0.38 g, 1.19 mmol). LC-MS (API-ES, pos.) M+H⁺ 319.0; ¹H NMR (CDCl₃) 7.56-7.62 (2H, m), 7.42-7.48 (3H, m), 7.28 (1H, d, J=8.5 Hz), 6.55 (1H, d, J=8.5 Hz), 3.18-3.30 (1H, m), 1.28-2.02 (10H, m) ppm.

Step 2. A mixture of 6-cyclohexylsulfanyl-3-phenylsulfanyl-pyridazine-1-oxide (0.38 g, 1.19 mmol) and mcpba (1.76 g, 7.14 mmol, 70%) in methylene chloride (50 mL) was stirred at ambient temperature over night. The solution was washed with saturated aqueous sodium bicarbonate solution and evaporated to afford a residue which was subjected to column chromatography on silica gel (eluent, hexane:ethyl acetate, 80:20 to 60:40. Some fractions containing the desired material showed the formation of crystalline material which was separated by filtration to afford 3-benzenesulfonyl-6-cyclohexanesulfonyl-pyridazine-1-oxide (0.05 g, 0.13 mmol). LC-MS (API-ES, pos.) M+H⁺ 383.0; ¹H NMR (CDCl₃) 8.52 (1H, d, J=8.1 Hz), 8.06-8.12 (2H, m), 7.96 (1H, d, J=8.1 Hz), 7.72-7.79 (1H, m), 7.60-7.66 (2H, m), 3.91-4.03 (1H, m), 1.12-1.97 (10H, m) ppm.

The LC₅₀ value for this compound in A549 and HL-60 cell lines were 278.7 μM and 32.9 μM respectively.

### EXAMPLE 6

In Vitro Studies

The compounds provided herein were tested for acute cytoxicity (apoptotic activity) using two human cancer cell lines expressing high levels of either Bcl-2 or BCl-X_{L}. HL-60 cells expressing high Bcl-2 and low Bcl-X_{L} levels, and A549 human lung cells expressing low Bcl-2 and high Bcl-X_{L} levels were incubated with the compounds provided herein at concentrations between 0.5 and 1000 μg/mL for 4 hours at 37° C., 5% CO₂. The number of viable cells were measured by mitochondrial transformation of Alamar Blue to a fluorescent dye. As negative controls, the compounds were tested against the cell lines with low levels of Bcl-2 expression, viz., MDA-453 cells. LC₅₀ values for each compound and controls were determined.

Since modifications will be apparent to those of skill in this art, it is intended that the subject matter claimed herein be limited only by the scope of the appended claims.

We claim:

1. The compound of Formula 8, 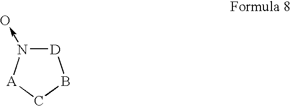
or a pharmaceutically acceptable derivative thereof, wherein
A is selected from 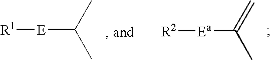
B is selected from 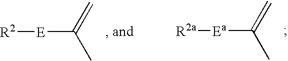
C is selected from 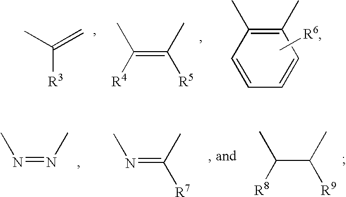
D is selected from —N—, —NO—, —NR¹⁰ , —CR¹¹R¹²—, —CR¹³—, —S—, —SO—, and —SO₂—;
E and E^{a} are each independently selected from single bond, —CR¹⁴R¹⁵, —NR¹⁶, —O—, —S—, —SO—, and —SO₂;
R¹ to R⁵, R⁷ to R ¹⁶, and R^{2a} are each independently selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C1-C10 alkylsulfonylalkyl, C1-C10 alkylcarbonylaminoC1-C10alkyl, C₃-C₂₀ heterocyclyl, C₃-C₂₀ heterocyclylalkyl, C₅-C₂₀ aralkyl, C₅-C₂₀ aryl, C₅-C₂₀ heteroaralkyl and C₅-C₂₀ heteroaryl, wherein the aryl and heteroaryl groups are optionally substituted with one or more halogen, trihaloalkyl, cyano, nitro, carboxy, alkoxycarbonyl, hydroxyl, alkoxy, acyloxyl, amino, alkylamino, acylamino, mercapto, or alkylthio, with the proviso that when C is 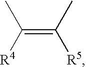
and E and E^{a} are both SO₂, then at least one of R² and R^{2a} is not CH₃; and
R⁶ is selected from hydrogen; electron donating groups such as C1-C10 alkyl, C3-C10 cycloalkyl, hydroxyl, C1-C10 alkoxy, amino, C1-C10 acylamino, mercapto or C1-C10 alkylthio; and electron withdrawing groups such as C1-C10 acyl, halo, mono- or polyhaloalkyl, cyano, nitro, carboxy, C1-C10 alkoxycarbonyl, C1-C10 alkylsulfonyl, C5-C10 aryl, C1-C10 alkoxyalkyl, C1-C10 aminoalkyl, C1-C10 alkylaminoalkyl, C1-C10 hydroxyalkyl, C1-C10 carboxyalkyl, C1-C10 alkoxycarbonylalkyl, C1-C10 mercaptoalkyl, C1-C10 alkylthioalkyl, C1-C10 sulfonylalkyl, and C1-C10 alkylsulfonylalkyl.

2. The compound of claim 1, wherein 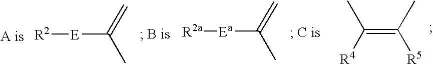
D is —N—, —NO—, or —CR¹³—;
E and E^{a} are each independently selected from a single bond, —CR¹⁴R¹⁵, NR¹⁶, —O—,—S—, —SO—, and —SO₂;
R², R^{2a}, R⁴, R⁵ and R¹³ to R¹⁶, are each independently selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C1-C10 alkylsulfonylalkyl, C1-C10 alkylcarbonylaminoC1-C10alkyl, C₃-C₂₀ heterocyclyl, C₃-C₂₀ heterocyclylalkyl, C₅-C₂₀ aralkyl, C₅-C₂₀ aryl, C₅-C₂₀ heteroaralkyl and C₅-C₂₀ heteroaryl, wherein aryl and heteroaryl groups are optionally substituted with one or more halogen, trihaloalkyl, cyano, nitro, carboxy or alkoxycarbonyl, hydroxyl, alkoxy, acyloxyl, amino, alkylamino, acylamino, mercapto, or alkylthio, with the proviso that when E and E^{a} are both SO₂, then at least one of R² and R^{2a} is not CH₃.

3. The compound of claim 1, wherein 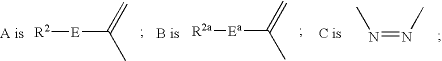
D is —N—, —NO—; E and E^{a} are each independently selected from single bond, —O—, —S—, —SO—, and —SO₂; R² and R^{2a} are each independently selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C1-C10 alkylsulfonylalkyl, C1-C10 alkylcarbonylaminoC1-C10alkyl, C₃-C₂₀ heterocyclyl, C₃-C₂₀ heterocyclylalkyl, C₅-C₂₀ aralkyl, C₅-C₂₀ aryl, C₅-C₂₀ heteroaralkyl and C₅-C₂₀ heteroaryl, wherein the aryl and heteroaryl groups are optionally substituted with one or more halogen, trihaloalkyl, cyano, nitro, carboxy, alkoxycarbonyl, hydroxyl, alkoxy, acyloxyl, amino, alkylamino, acylamino, mercapto, or alkylthio.

4. The compound of claim 1, wherein 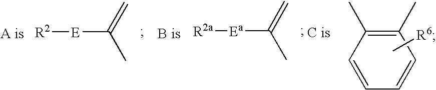
D is —N—, —NO—; E and E^{a} are each independently selected from a single bond, —O—, —S—, —SO—, and —SO₂; R² and R^{2a} are each independently selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C₅-C₂₀ aryl, and C₅-C₂₀ heteroaryl, wherein the aryl and heteroaryl groups are optionally substituted with one or more halogen, trihaloalkyl, cyano, nitro, carboxy, alkoxycarbonyl, hydroxyl, alkoxy, acyloxyl, amino, alkylamino, acylamino, mercapto, or alkylthio; and R₆ is selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl.

5. The compound of claim 1, wherein A is 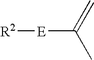
wherein R² is selected from hydrogen, C1-C10 alkyl, C3-C10 cycloalkyl, C 1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C1-C10 alkylsulfonylalkyl, C1-C10 alkylcarbonylaminoC1-C10 alkyl, C₃-C₂₀ heterocyclyl, C₃-C₂₀ heterocyclylalkyl, C5-C20 aralkyl; C5-C20 aryl, C5-C20 heteroaralkyl and C5-C20 heteroaryl, wherein the aryl and heteroaryl groups are optionally substituted with one or more halogen, trihaloalkyl, cyano, nitro, carboxy, alkoxycarbonyl, hydroxyl, alkoxy, acyloxyl, amino, alkylamino, acylamino, mercapto, or alkylthio.

6. The compound of claim 1, wherein B is 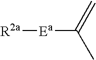
where R^{2a} is selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C1-C10 alkylsulfonylalkyl, C1-C10 alkylcarbonylaminoC1-C10alkyl, C₃-C₂₀ heterocyclyl, C₃-C₂₀ heterocyclylalkyl, C5-C20 aralkyl, C5-C20 aryl, C5-C20 heteroaralkyl and C5-C20 heteroaryl, wherein the aryl and heteroaryl groups are optionally substituted with one or more halogen, trihaloalkyl, cyano, nitro, carboxy, alkoxycarbonyl, hydroxyl, alkoxy, acyloxyl, amino, alkylamino, acylamino, mercapto, or alkylthio.

7. The compound of claim 1, where R² and R^{2a} are each independently selected from halogen, C1-C10 alkyl, C3-C10 cycloalkyl, C1-C10 hydroxyalkyl, C1-C10 alkoxycarbonylalkyl, C1-C10 alkylcarbonylaminoC1-C10alkyl, C₃-C₂₀ heterocyclyl, C₃-C₂₀ heterocyclylalkyl, C₅-C₂₀ aralkyl, C₅-C₂₀ aryl, C₅-C₂₀ heteroaralkyl and C₅-C₂₀ heteroaryl, where the aryl and heteroaryl groups are optionally substituted with one or more groups selected from alkyl, haloalkyl, halogen, alkoxycarbonyl, carboxy, hydroxy, alkylsulfonylamino, alkoxy and nitro.

8. The compound of claim 1, where R² and R^{2a} are each independently selected from chloro, methyl, ethyl, n-butyl, tert-butyl, hydroxyethyl, methoxycarbonylethyl, methylcarbonylaminoethyl, cyclopentyl, cyclohexyl, benzyl, phenyl, naphthyl, N-morpholinyl, 2-pyridinyl and 4-pyridinyl, where the phenyl and pyridinyl rings are optionally substituted with one or more groups selected from methyl, trifluoromethyl, chloro, methoxycarbonyl, carboxy, hydroxy, methylsulfonylamino, methoxy and nitro.

9. The compound of claim 1, where R² and R^{2a} are each independently selected from chloro, methyl, ethyl, n-butyl, tert-butyl, hydroxyethyl, methoxycarbonylethyl, methylcarbonylaminoethyl cyclopentyl, cyclohexyl, N-morpholinyl, 4-pyridinyl, benzyl, phenyl, 2-pyridinyl, 4-nitrophenyl, 4-methoxyphenyl, p-tolyl, 4-trifluoromethyl, m-tolyl, o-tolyl, 3-methoxyphenyl, 2-methoxyphenyl, 2-chlorophenyl, naphthyl, 2-methoxycarbonylphenyl, 2-carboxyphenyl, 4-hydroxyphenyl, 4-methoxycarbonylphenyl, 4-aminophenyl, 4-carboxyphenyl and 4-methylsulfonylaminophenyl.

10. The compound of claim 1, where R² is chloro, methyl, ethyl, n-butyl, tert-butyl, hydroxyethyl, acetoxyethyl, methylcarbonylaminoethyl cyclopentyl, cyclohexyl, N-morpholinyl, 4-pyridinyl, benzyl, phenyl, 2-pyridinyl, 4-nitrophenyl, 4-methoxyphenyl, p-tolyl, 4-trifluoromethyl, m-tolyl, o-tolyl, 3-methoxyphenyl, 2-methoxyphenyl, 2-chlorophenyl, naphthyl, 2-methoxycarbonylphenyl, 2-carboxyphenyl, 4-hydroxyphenyl, 4-methoxycarbonylphenyl, 4-aminophenyl, 4-carboxyphenyl and 4-, methylsulfonylaminophenyl.

11. The compound of claim 1, where R^{2a} is chloro, methyl, ethyl, n-butyl, tert-butyl, hydroxyethyl, methoxycarbonylethyl, methylcarbonylaminoethyl cyclopentyl, cyclohexyl, N-morpholinyl, 4-pyridinyl, benzyl, phenyl, 2-pyridinyl, 4-nitrophenyl, 4-methoxyphenyl, p-tolyl, 4-trifluoromethyl, m-tolyl, o-tolyl, 3-methoxyphenyl, 2-methoxyphenyl, 2-chlorophenyl, naphthyl, 2-methoxycarbonylphenyl, 2-carboxyphenyl, 4-hydroxyphenyl, 4-methoxycarbonylphenyl, 4-aminophenyl, 4-carboxyphenyl and 4-methylsulfonylaminophenyl.

12. The compound of claim 1, where C is selected from a group consisting of 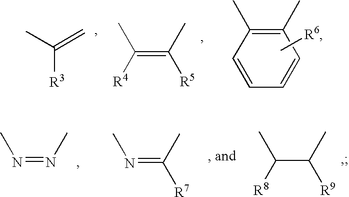
wherein R³to R⁵ and R⁷ to R⁹ are each independently selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C1-C10 alkylsulfonylalkyl,C5-C20 aralkyl, C5-C20 aryl, C5-C20 heteroaralkyl and C5-C20 heteroaryl, wherein the aryl and heteroaryl groups are optionally substituted with one or more halogen, trihaloalkyl, cyano, nitro, carboxy, alkoxycarbonyl, hydroxyl, alkoxy, acyloxyl, amino, alkylamino, acylamino, mercapto, or alkylthio and R⁶ is selected from hydrogen; C1-C10 alkyl, C3-C10 cycloalkyl, hydroxyl, C1-C10 alkoxy, amino, C1-C10 acylamino, mercapto, C1-C10 alkylthio; C1-C10 acyl, halo, mono- or polyhaloalkyl, cyano, nitro, carboxy, C1-C10 alkoxycarbonyl, C1-C10 alkylsulfonyl, C5-C10 aryl, C1-C10 alkoxyalkyl, C1-C10 aminoalkyl, C1-C10 alkylaminoalkyl, C1-C10 hydroxyalkyl, C1-C10 carboxyalkyl, C1-C10 alkoxycarbonylalkyl, C1-C10 mercaptoalkyl, C1-C10 alkylthioalkyl, C1-C10 sulfonylalkyl, and C1-C10 alkylsulfonylalkyl.

13. The compound of claim 1, where C is 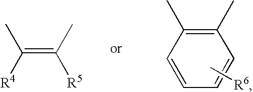
wherein R⁴ and R⁵ are each independently hydrogen, C1-C10 alkyl or C3-C10 cycloalkyl, and R⁶ is hydrogen or C1-C10 alkyl.

14. The compound of claim 1, where R⁴ and R⁵ are each independently selected from hydrogen and C1-C10 alkyl.

15. The compound of claim 1, where R⁴ and R⁵ are each independently hydrogen or methyl.

16. The compound of claim 1, where R⁴ is hydrogen or methyl.

17. The compound of claim 1, where R⁴ is methyl.

18. The compound of claim 1, where R⁵is hydrogen.

19. The compound of claim 1, where C is 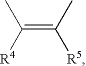
wherein R⁴ and R⁵ are each independently hydrogen or methyl.

20. The compound of claim 1, where
C is

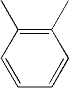

21. The compound of claim 1, where D is selected from —N—, —NO—, —NR¹⁰, —CR¹¹R¹²—, —CR¹³—, —S—, —SO—, and —SO₂.

22. The compound of claim 1, where D is —N— or —NO—.

23. The compound of claim 1, where D is —N—.

24. The compound of claim 1, where E and E^{a} are each independently selected from a single bond, —CR¹⁴R¹⁵, —NR¹⁶, —O—, —S—, —SO—, and —SO₂—.

25. The compound of claim 1, where E^{a} is single bond, —O—, —SO—, or —SO₂—.

26. The compound of claim 1, where E is single bond, —O—, —SO—, or —SO₂—.

27. The compound of claim 1, where E^{a} is —SO₂—.

28. The compound of claim 1, where E is —SO₂—.

29. The compound of claim 1, where R⁶ is hydrogen.

30. The compound of claim 1 that has formula: 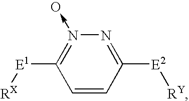
wherein E¹ and E² are each independently selected from a single bond, —O—, —S—, —SO— or —SO₂—; R^{x} and R^{y} are each independently selected from halogen, C1-C10 alkyl, C3-C10 cycloalkyl, C1-C10 hydroxyalkyl, C1-C10 alkoxycarbonylalkyl, C1-C10 alkylcarbonylaminoC1-C10alkyl, C₃-C₂₀ heterocyclyl, C₃-C₂₀ heterocyclylalkyl, C₅-C₂₀ aralkyl, C₅-C₂₀ aryl, C₅-C₂₀ heteroaralkyl and C₅-C₂₀ heteroaryl, where the aryl and heteroaryl groups are optionally substituted with one or more groups selected from alkyl, haloalkyl, halogen, alkoxycarbonyl, carboxy, hydroxy, alkylsulfonylamino, alkoxy and nitro, with the proviso that when and E and E^{a} are both SO₂, then at least one of R^{x} and R^{y} is not CH₃.

31. The compound of claim 30, where E¹ is chloro, —O— or —SO₂—.

32. The compound of claim 30, where E² is chloro, —O— or —SO₂—.

33. The compound of claim 30, where R^{x} and R^{y} are each independently selected from chloro, methyl, ethyl, n-butyl, tert-butyl, hydroxyethyl, methoxycarbonylethyl, methylcarbonylaminoethyl, cyclopentyl, cyclohexyl, benzyl, phenyl, naphthyl, N-morpholinyl, 2-pyridinyl and 4-pyridinyl, where the phenyl and pyridinyl rings are optionally substituted with one or more groups selected from methyl, trifluoromethyl, chloro, methoxycarbonyl, carboxy, hydroxy, methylsulfonylamino, methoxy and nitro.

34. The compound of claim 30, where R^{x} is phenyl, optionally substituted with one or more methyl, trifluoromethyl, chloro, methoxycarbonyl, carboxy, hydroxy, methylsulfonylamino, methoxy or nitro.

35. The compound of claim 30, where R^{y} is phenyl, optionally substituted with one or more methyl, trifluoromethyl, chloro, methoxycarbonyl, carboxy, hydroxy, methylsulfonylamino, methoxy or nitro.

36. The compound of claim 30 that has formula:

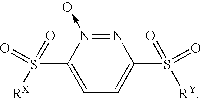

37. The compound of claim 36 that has formula: 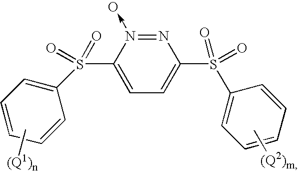
wherein m and n are each independently an integer from 0 to 4, and Q¹ and Q² are each independently selected from methyl, trifluoromethyl, chloro, methoxycarbonyl, carboxy, hydroxy, methylsulfonylamino, methoxy and nitro.

38. The compound of claim 30 that is selected from 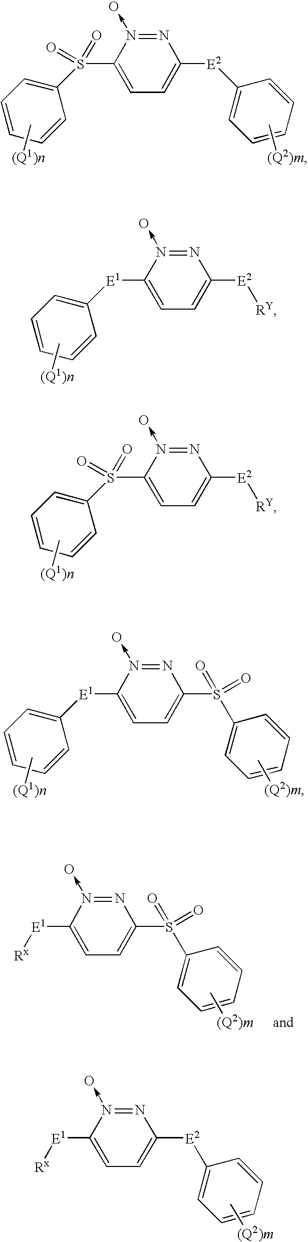
wherein m and n are each independently an integer from 0 to 4, and Q¹ and Q² are each independently selected from methyl, trifluoromethyl, chloro, methoxycarbonyl, carboxy, hydroxy, methylsulfonylamino, methoxy and nitro.

39. The compound of claim 38 wherein m is 0 or 1.

40. The compound of claim 38 wherein n is 0 or 1.

41. The compound of claim 36 that has formula 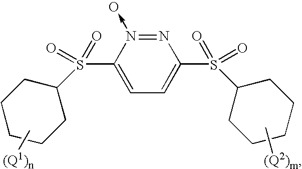
wherein m and n are each independently an integer from 0 to 4, and Q¹ and Q² are each independently selected from methyl, trifluoromethyl, chloro, methoxycarbonyl, carboxy, hydroxy, methylsulfonylamino, methoxy and nitro.

42. The compound of claim 30 that has formula selected from

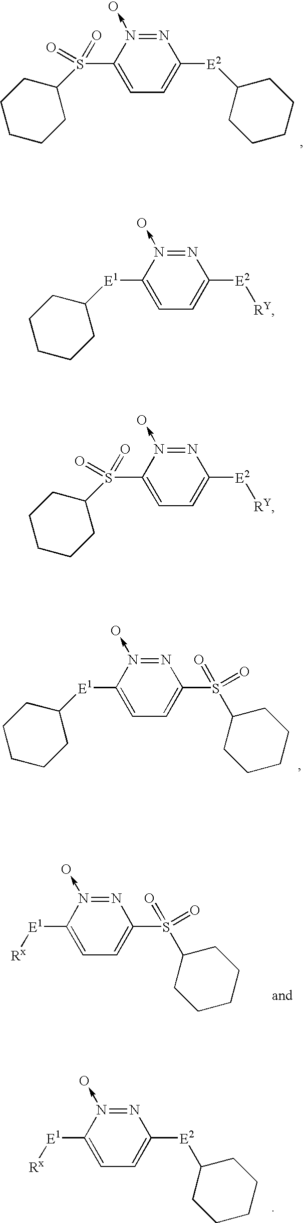

43. A compound of claim 1 that is selected from

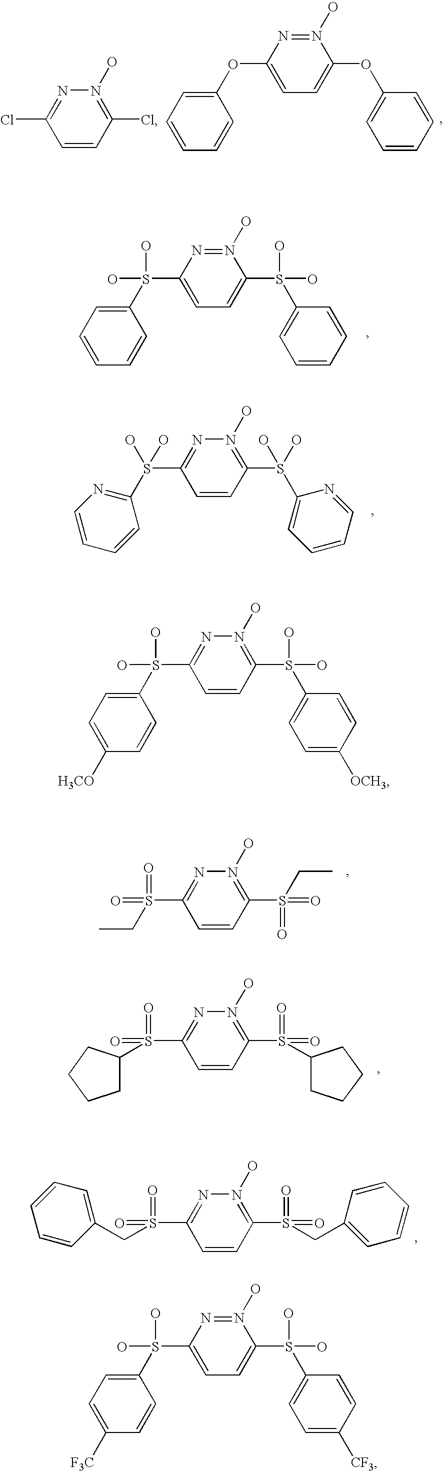

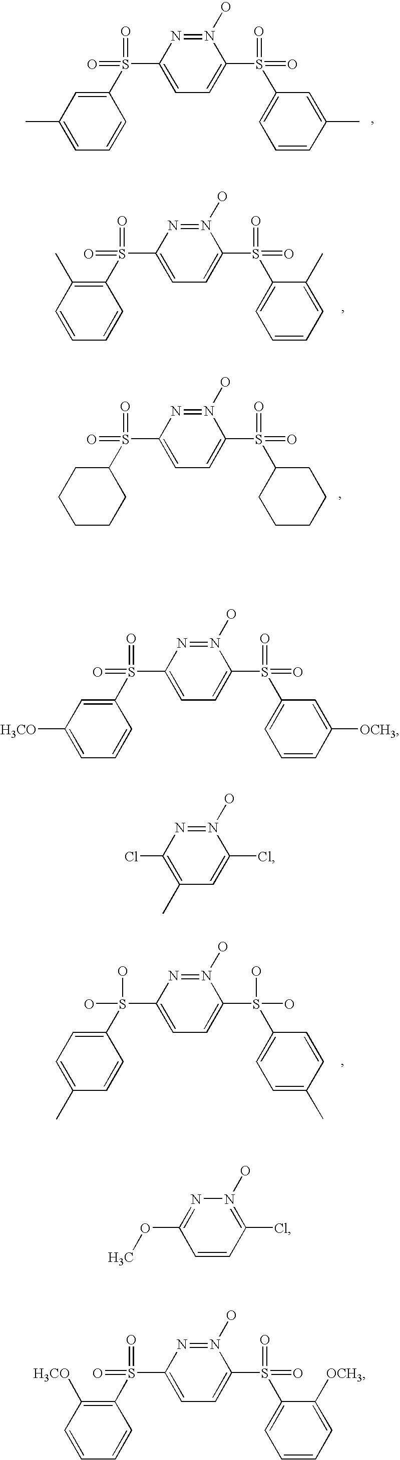

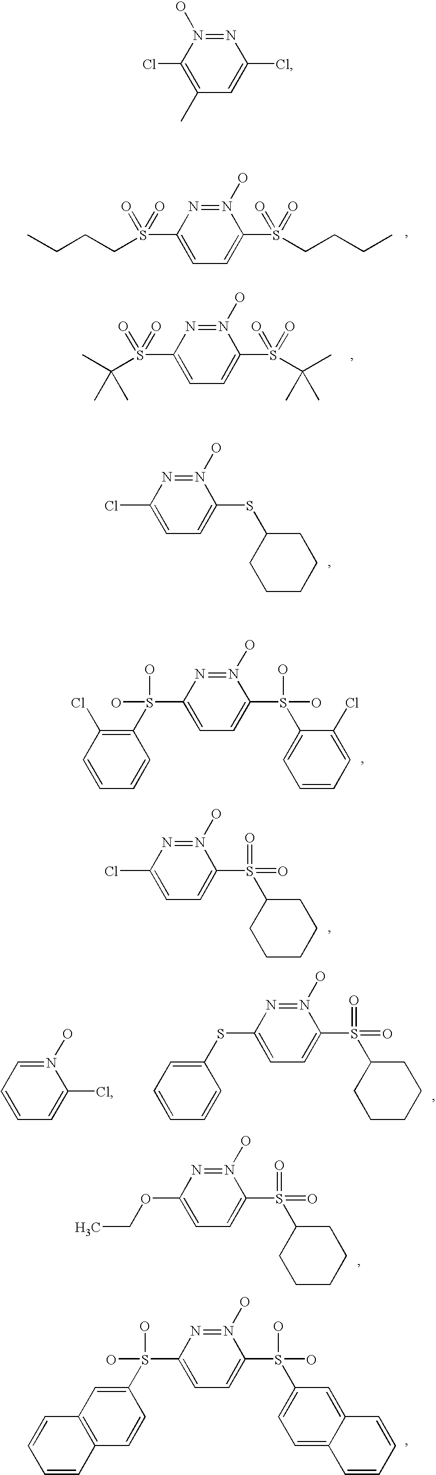

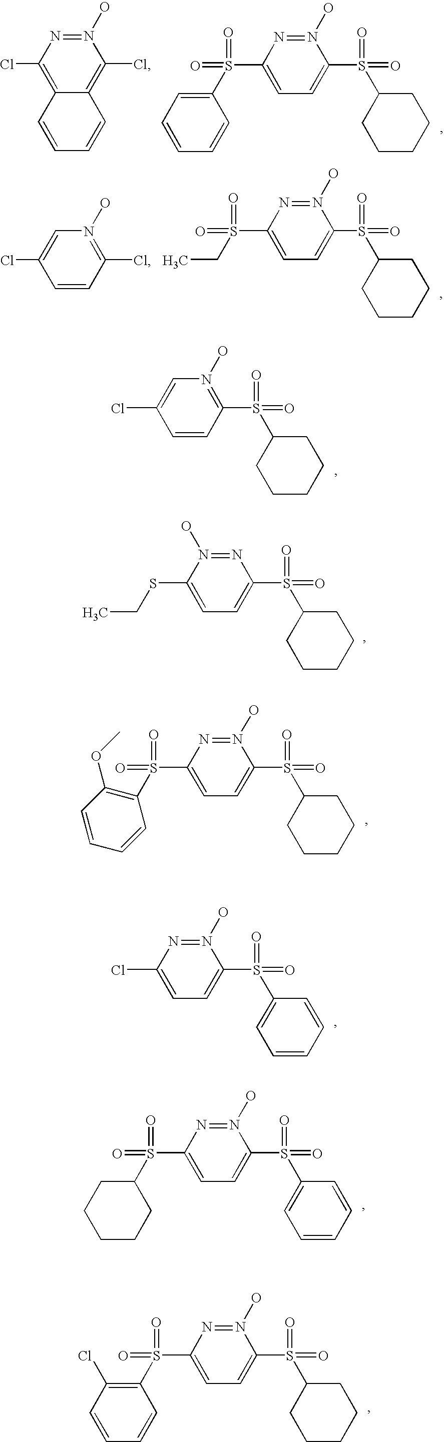

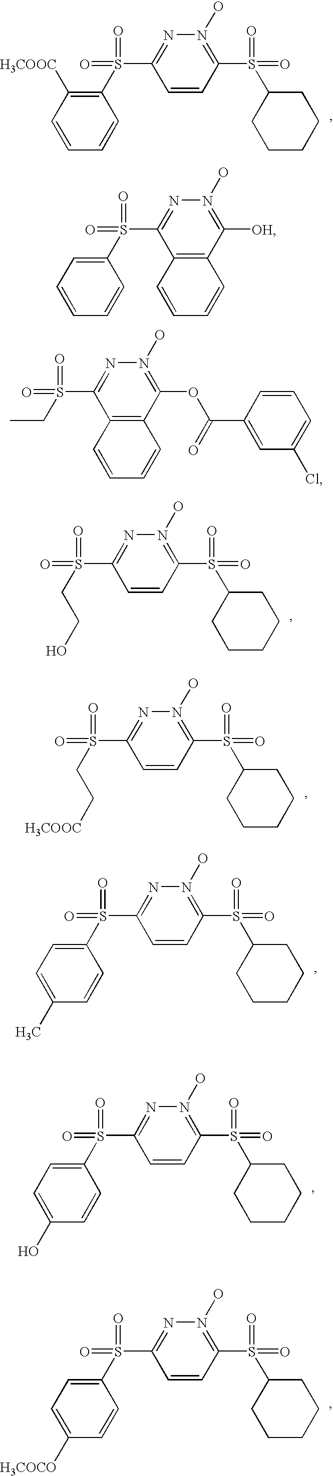

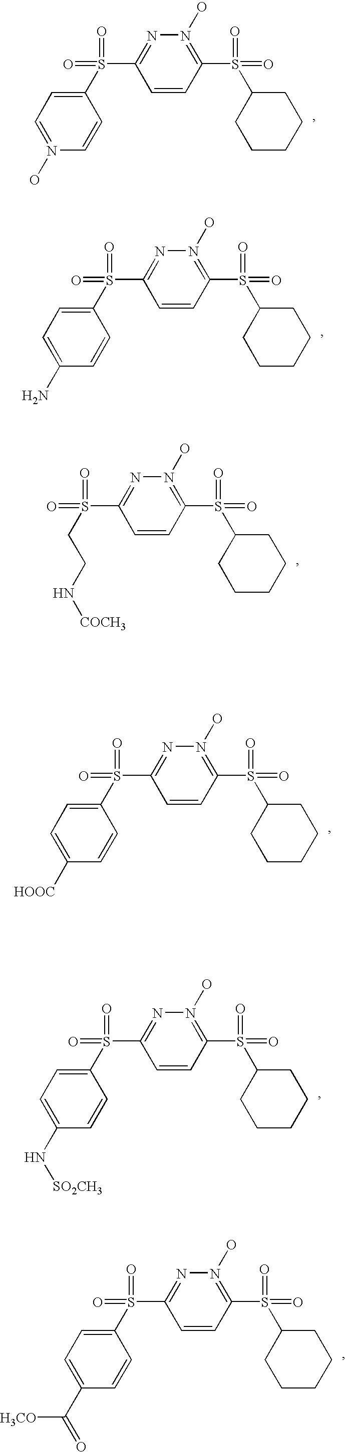

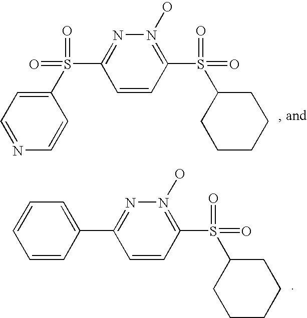

44. A pharmaceutical composition, comprising, in a pharmaceutically acceptable carrier, a compound of formula 8, 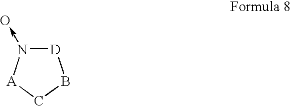
or a pharmaceutically acceptable derivative thereof, wherein
A is selected from 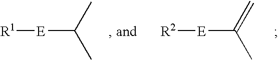
B is selected from 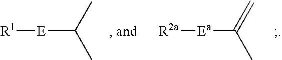
C is selected from 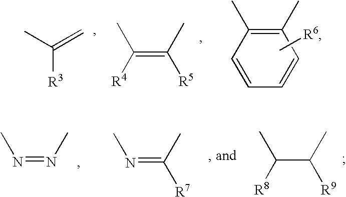
D is selected from —N—, —NO—, —NR¹⁰, —CR¹¹R¹²—, —CR¹³—, —S—, —SO—, and —SO₂—;
E and E^{a} are each independently selected from single bond, —CR¹⁴R¹⁵, —NR¹⁶, —O—, —S—, —SO—, and —SO₂;
R¹ to R⁵, R⁷ to R¹⁶, and R^{2a} are each independently selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C1-C10 alkylsulfonylalkyl, C1-C10 alkylcarbonylaminoC1-C10alkyl, C₃-C₂₀ heterocyclyl, C₃-C₂₀ heterocyclylalkyl, C₅-C₂₀ aralkyl, C₅-C₂₀ aryl, C₅-C₂₀ heteroaralkyl and C₅-C₂₀ heteroaryl, wherein the aryl and heteroaryl groups are optionally substituted with one or more halogen, trihaloalkyl, cyano, nitro, carboxy, alkoxycarbonyl, hydroxyl, alkoxy, acyloxyl, amino, alkylamino, acylamino, mercapto, or alkylthio, with the proviso that when C is 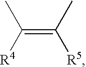
and E and E^{a} are both SO₂, then at least one of R² and R^{2a} is not CH₃; and
R⁶ is selected from hydrogen; electron donating groups such as C1-C10 alkyl, C3-C10 cycloalkyl, hydroxyl, C1-C10 alkoxy, amino, C1-C10 acylamino, mercapto or C1-C10 alkylthio; and electron withdrawing groups such as C1-C10 acyl, halo, mono- or polyhaloalkyl, cyano, nitro, carboxy, C1-C10 alkoxycarbonyl, C1-C10 alkylsulfonyl, C5-C10 aryl, C1-C10 alkoxyalkyl, C1-C10 aminoalkyl, C1-C10 alkylaminoalkyl, C1-C10 hydroxyalkyl, C1-C10 carboxyalkyl, C1-C10 alkoxycarbonylalkyl, C1-C10 mercaptoalkyl, C1-C10 alkylthioalkyl, C1-C10 sulfonylalkyl, and C1-C10 alkylsulfonylalkyl.

45. The pharmaceutical composition of claim 44, wherein 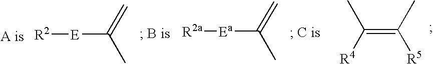
D is —N—, —NO—, or —CR¹³—;
E and E^{a} are each independently selected from a single bond, —CR¹⁴R¹⁵, NR¹⁶, —O—, —S—, —SO—, and —SO₂;
R², R^{2a}, R⁴, R⁵ and R¹³ to R¹⁶, are each independently selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C1-C10 alkylsulfonylalkyl, C1-C10 alkylcarbonylaminoC1-C10alkyl, C₃-C₂₀ heterocyclyl, C₃-C₂₀ heterocyclylalkyl, C₅-C₂₀ aralkyl, C₅-C₂₀ aryl, C₅-C₂₀ heteroaralkyl and C₅-C₂₀ heteroaryl, wherein aryl and heteroaryl groups are optionally substituted with one or more halogen, trihaloalkyl, cyano, nitro, carboxy or alkoxycarbonyl, hydroxyl, alkoxy, acyloxyl, amino, alkylamino, acylamino, mercapto, or alkylthio with the proviso that when E and E^{a} are both SO₂, then at least one of R² and R^{2a} is not CH₃.

46. The pharmaceutical composition of claim 44, wherein 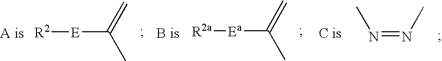
D is —N—, —NO—; E and E^{a} are each independently selected from single bond, —O—, —S—, —SO—, and —SO₂; R² and R^{2a} are each independently selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C1-C10 alkylsulfonylalkyl, C1-C10 alkylcarbonylaminoC1-C10alkyl, C₃-C₂₀ heterocyclyl, C₃-C₂₀ heterocyclylalkyl, C₅-C₂₀ aralkyl, C₅-C₂₀ aryl, C₅-C₂₀ heteroaralkyl and C₅-C₂₀ heteroaryl, wherein the aryl and heteroaryl groups are optionally substituted with one or more halogen, trihaloalkyl, cyano, nitro, carboxy, alkoxycarbonyl, hydroxyl, alkoxy, acyloxyl, amino, alkylamino, acylamino, mercapto, or alkylthio.

47. The pharmaceutical composition of claim 44, wherein 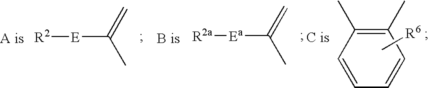
D is —N—, —NO—; E and E^{a} are each independently selected from a single bond, —O—, —S—, —SO—, and —SO₂; R² and R^{2a} are each independently selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C₅-C₂₀ aryl, and C₅-C₂₀ heteroaryl, wherein the aryl and heteroaryl groups are optionally substituted with one or more halogen, trihaloalkyl, cyano, nitro, carboxy, alkoxycarbonyl, hydroxyl, alkoxy, acyloxyl, amino, alkylamino, acylamino, mercapto, or alkylthio; and R⁶ is selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl.

48. The pharmaceutical composition of claim 44, wherein A is 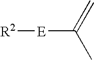
wherein R² is selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C1-C10 alkylsulfonylalkyl, C1-C10 alkylcarbonylaminoC1-C10alkyl, C₃-C₂₀ heterocyclyl, C₃-C₂₀ heterocyclylalkyl, C5-C20 aralkyl, C5-C20 aryl, C5-C20 heteroaralkyl and C5-C20 heteroaryl, wherein the aryl and heteroaryl groups are optionally substituted with one or more halogen, trihaloalkyl, cyano, nitro, carboxy, alkoxycarbonyl, hydroxyl, alkoxy, acyloxyl, amino, alkylamino, acylamino, mercapto, or alkylthio.

49. The pharmaceutical composition of claim 44, wherein B is 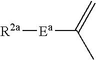
where R^{2a} is selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C1-C10 alkylsulfonylalkyl, C1-C10 alkylcarbonylaminoC1-C10alkyl, C₃-C₂₀ heterocyclyl, C₃-C₂₀ heterocyclylalkyl, C5-C20 aralkyl, C5-C20 aryl, C5-C20 heteroaralkyl and C5-C20 heteroaryl, wherein the aryl and heteroaryl groups are optionally substituted with one or more halogen, trihaloalkyl, cyano, nitro, carboxy, alkoxycarbonyl, hydroxyl, alkoxy, acyloxyl, amino, alkylamino, acylamino, mercapto, or alkylthio.

50. The pharmaceutical composition of claim 44, where R² and R^{2a} are each independently selected from halogen, C1-C10 alkyl, C3-C10 cycloalkyl, C1-C10 hydroxyalkyl, C1-C10 alkoxycarbonylalkyl, C1-C10 alkylcarbonylaminoC1-C10alkyl, C₃-C₂₀ heterocyclyl, C₃-C₂₀ heterocyclylalkyl, C₅-C₂₀ aralkyl, C₅-C₂₀ aryl, C₅-C₂₀ heteroaralkyl and C₅-C₂₀ heteroaryl, where the aryl and heteroaryl groups are optionally substituted with one or more groups selected from alkyl, haloalkyl, halogen, alkoxycarbonyl, carboxy, hydroxy, alkylsulfonylamino, alkoxy and nitro.

51. The pharmaceutical composition of claim 44, where R² and R^{2a} are each independently selected from chloro, methyl, ethyl, n-butyl, tert-butyl, hydroxyethyl, methoxycarbonylethyl, methylcarbonylaminoethyl, cyclopentyl, cyclohexyl, benzyl, phenyl, naphthyl, N-morpholinyl, 2-pyridinyl and 4-pyridinyl, where the phenyl and pyridinyl rings are optionally substituted with one or more groups selected from methyl, trifluoromethyl, chloro, methoxycarbonyl, carboxy, hydroxy, methylsulfonylamino, methoxy and nitro.

52. The pharmaceutical composition of claim 44, where R² and R^{2a} are each independently selected from chloro, methyl, ethyl, n-butyl, tert-butyl, hydroxyethyl, methoxycarbonylethyl, methylcarbonylaminoethyl cyclopentyl, cyclohexyl, N-morpholinyl, 4-pyridinyl, benzyl, phenyl, 2-pyridinyl, 4-nitrophenyl, 4-methoxyphenyl, p-tolyl, 4-trifluoromethyl, m-tolyl, o-tolyl, 3-methoxyphenyl, 2-methoxyphenyl, 2-chlorophenyl, naphthyl, 2-methoxycarbonylphenyl, 2-carboxyphenyl, 4-hydroxyphenyl, 4-methoxycarbonylphenyl, 4-aminophenyl, 4-carboxyphenyl and 4-methylsulfonylaminophenyl.

53. The pharmaceutical composition of claim 44, where R² is chloro, methyl, ethyl, n-butyl, tert-butyl, hydroxyethyl, methoxycarbonylethyl, methylcarbonylaminoethyl cyclopentyl, cyclohexyl, N-morpholinyl, 4-pyridinyl, benzyl, phenyl, 2-pyridinyl, 4-nitrophenyl, 4-methoxyphenyl, p-tolyl, 4-trifluoromethyl, m-tolyl, o-tolyl, 3-methoxyphenyl, 2-methoxyphenyl, 2-chlorophenyl, naphthyl, 2-methoxycarbonylphenyl, 2-carboxyphenyl, 4-hydroxyphenyl, 4-methoxycarbonylphenyl, 4-aminophenyl, 4-carboxyphenyl and 4-methylsulfonylaminophenyl.

54. The pharmaceutical composition of claim 44, where R^{2a} is chloro, methyl, ethyl, n-butyl, tert-butyl, hydroxyethyl, methoxycarbonylethyl, methylcarbonylaminoethyl cyclopentyl, cyclohexyl, N-morpholinyl, 4-pyridinyl, benzyl, phenyl, 2-pyridinyl, 4-nitrophenyl, 4-methoxyphenyl, p-tolyl, 4-trifluoromethyl, m-tolyl, o-tolyl, 3-methoxyphenyl, 2-methoxyphenyl, 2-chlorophenyl, naphthyl, 2-methoxycarbonylphenyl, 2-carboxyphenyl, 4-hydroxyphenyl, 4-methoxycarbonylphenyl, 4-aminophenyl, 4-carboxyphenyl and 4-methylsulfonylaminophenyl.

55. The pharmaceutical composition of claim 44, where C is selected from a group consisting of 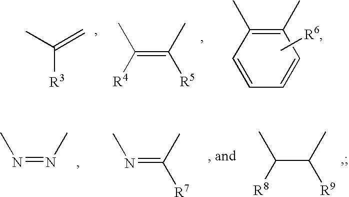
wherein R³ to R⁵ and R⁷ to R⁹ are each independently selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C1-C10 alkylsulfonylalkyl,C5-C20 aralkyl, C5-C20 aryl, C5-C20 heteroaralkyl and C5-C20 heteroaryl, wherein the aryl and heteroaryl groups are optionally substituted with one or more halogen, trihaloalkyl, cyano, nitro, carboxy, alkoxycarbonyl, hydroxyl, alkoxy, acyloxyl, amino, alkylamino, acylamino, mercapto, or alkylthio and R⁶ is selected from hydrogen; C1-C10 alkyl, C3-C10 cycloalkyl, hydroxyl, C1-C10 alkoxy, amino, C1-C10 acylamino, mercapto, C1-C10 alkylthio; C1-C10 acyl, halo, mono- or polyhaloalkyl, cyano, nitro, carboxy, C1-C10 alkoxycarbonyl, C1-C10 alkylsulfonyl, C5-C10 aryl, C1-C10 alkoxyalkyl, C1-C10 aminoalkyl, C1-C10 alkylaminoalkyl, C1-C10 hydroxyalkyl, C1-C10 carboxyalkyl, C1-C10 alkoxycarbonylalkyl, C1-C10 mercaptoalkyl, C1-C10 alkylthioalkyl, C1-C10 sulfonylalkyl, and C1-C10 alkylsulfonylalkyl.

56. The pharmaceutical composition of claim 44, where C is 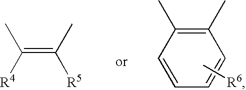
wherein R⁴ and R⁵ are each independently hydrogen, C1-C10 alkyl or C3-C10 cycloalkyl, and R⁶ is hydrogen or C1-C10 alkyl.

57. The pharmaceutical composition of claim 44, where R⁴ and R⁵ are each independently selected from hydrogen and C1-C10 alkyl.

58. The pharmaceutical composition of claim 44, where R⁴ and R⁵ are each independently hydrogen or methyl.

59. The pharmaceutical composition of claim 44, where R⁴ is hydrogen or methyl.

60. The pharmaceutical composition of claim 44, where R⁴ is methyl.

61. The pharmaceutical composition of claim 44, where R⁵ is hydrogen.

62. The pharmaceutical composition of claim 44, where C is 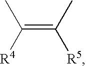
wherein R⁴ and R⁵ are each independently hydrogen or methyl.

63. The pharmaceutical composition of claim 44, where
C is

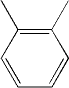

64. The pharmaceutical composition of claim 44, where D is selected from —N—, —NO—, —NR¹⁰, —CR¹¹R¹²—, —CR¹³—, —S—, —SO—, and —SO₂.

65. The pharmaceutical composition of claim 44, where D is —N—or —NO—.

66. The pharmaceutical composition of claim 44, where D is —N—.

67. The pharmaceutical composition of claim 44, where E and E^{a} are each independently selected from a single bond, —CR¹⁴R¹⁵, —NR¹⁶, —O—, —S—, —SO—, and —SO₂—.

68. The pharmaceutical composition of claim 44, where E^{a} is single bond,—O—, —SO—, or —SO₂—.

69. The pharmaceutical composition of claim 44, where E is single bond, —O—, —SO—, or —SO₂—.

70. The pharmaceutical composition of claim 44, where E^{a} is —SO₂—.

71. The pharmaceutical composition of claim 44, where E is —SO₂—.

72. The pharmaceutical composition of claim 44, where R⁶ is hydrogen.

73. The pharmaceutical composition of claim 44 that has formula: 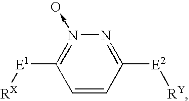
wherein E¹ and E² are each independently selected from a single bond, —O—, —S—, —SO—or —SO₂—; R^{x} and R^{y} are each independently selected from halogen, C1-C10 alkyl, C3-C10 cycloalkyl, C1-C10 hydroxyalkyl, C1-C10 alkoxycarbonylalkyl, C1-C10 alkylcarbonylaminoC1-C10alkyl, C₃-C₂₀ heterocyclyl, C₃-C₂₀ heterocyclylalkyl, C₅-C₂₀ aralkyl, C₅-C₂₀ aryl, C₅-C₂₀ heteroaralkyl and C₅-C₂₀ heteroaryl, where the aryl and heteroaryl groups are optionally substituted with one or more groups selected from alkyl, haloalkyl, halogen, alkoxycarbonyl, carboxy, hydroxy, alkylsulfonylamino, alkoxy and nitro, with the proviso that when and E and E^{a} are both SO₂, then at least one of R^{x} and R^{y} is not CH₃.

74. The pharmaceutical composition of claim 73, where E¹ is chloro, —O— or —SO₂—.

75. The pharmaceutical composition of claim 73, where E² is chloro, —O— or —SO₂—.

76. The pharmaceutical composition of claim 73, where R^{x} and R^{y} are each independently selected from chloro, methyl, ethyl, n-butyl, tert-butyl, hydroxyethyl, methoxycarbonylethyl, methylcarbonylaminoethyl, cyclopentyl, cyclohexyl, benzyl, phenyl, naphthyl, N-morpholinyl, 2-pyridinyl and 4-pyridinyl, where the phenyl and pyridinyl rings are optionally substituted with one or more groups selected from methyl, trifluoromethyl, chloro, methoxycarbonyl, carboxy, hydroxy, methylsulfonylamino, methoxy and nitro.

77. The pharmaceutical composition of claim 73, where R^{x} is phenyl, optionally substituted with one or more methyl, trifluoromethyl, chloro, methoxycarbonyl, carboxy, hydroxy, methylsulfonylamino, methoxy or nitro.

78. The pharmaceutical composition of claim 73, where R^{y} is phenyl, optionally substituted with one or more methyl, trifluoromethyl, chloro, methoxycarbonyl, carboxy, hydroxy, methylsulfonylamino, methoxy or nitro.

79. The pharmaceutical composition of claim 73 that has formula:

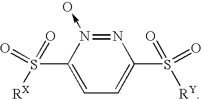

80. The pharmaceutical composition of claim 79 that has formula: 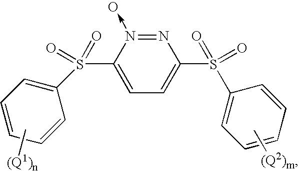
wherein m and n are each independently an integer from 0 to 4, and Q¹ and Q² are each independently selected from methyl, trifluoromethyl, chloro, methoxycarbonyl, carboxy, hydroxy, methylsulfonylamino, methoxy and nitro.

81. The pharmaceutical composition of claim 71 that is selected from 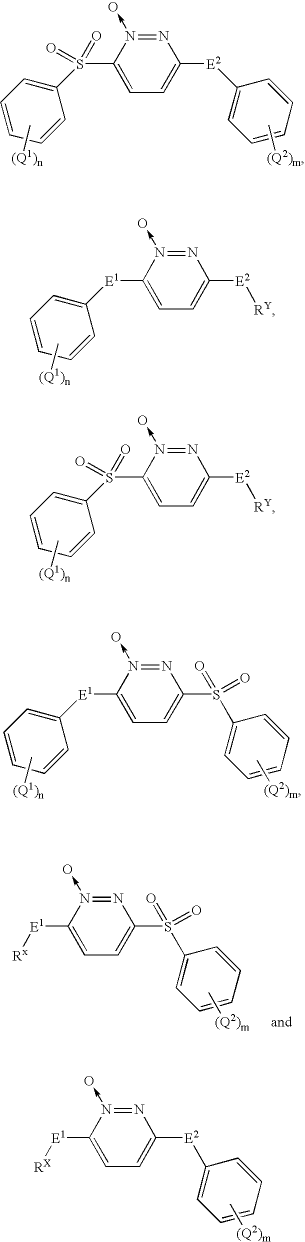
wherein m and n are each independently an integer from 0 to 4, and Q¹ and Q² are each independently selected from methyl, trifluoromethyl, chloro, methoxycarbonyl, carboxy, hydroxy, methylsulfonylamino, methoxy and nitro.

82. The pharmaceutical composition of claim 81 wherein m is 0 or 1.

83. The pharmaceutical composition of claim 81 wherein n is 0 or 1.

84. The pharmaceutical composition of claim 79 that has formula 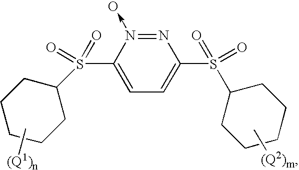
wherein m and n are each independently an integer from 0 to 4, and Q¹ and Q² are each independently selected from methyl, trifluoromethyl, chloro, methoxycarbonyl, carboxy, hydroxy, methylsulfonylamino, methoxy and nitro.

85. The pharmaceutical composition of claim 71 that has formula selected from

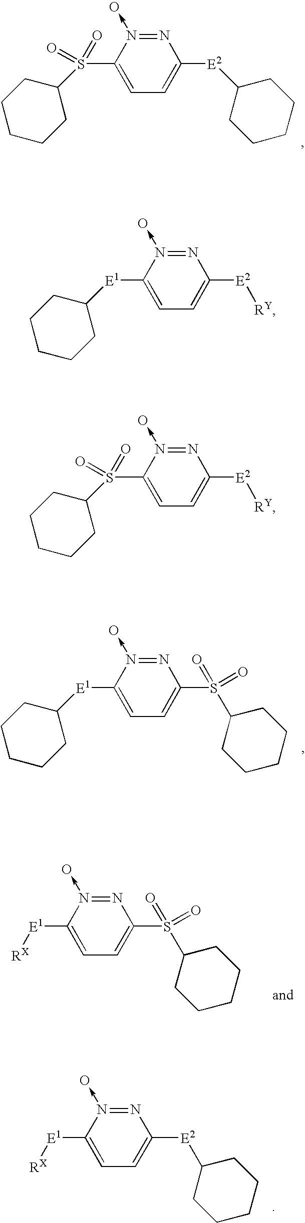

86. A pharmaceutical composition, comprising, in a pharmaceutically acceptable carrier, a compound selected from

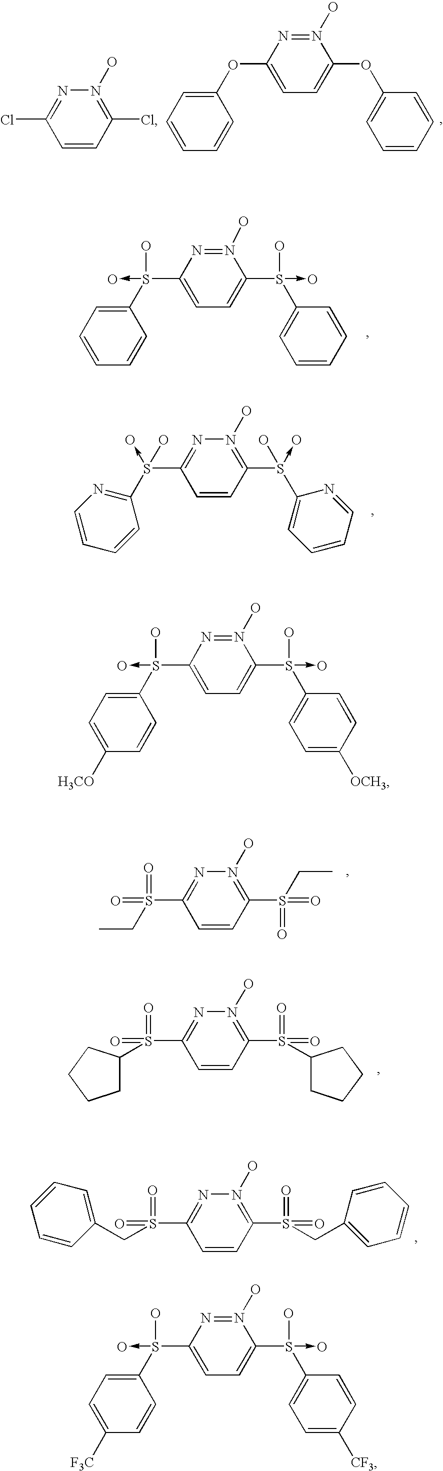

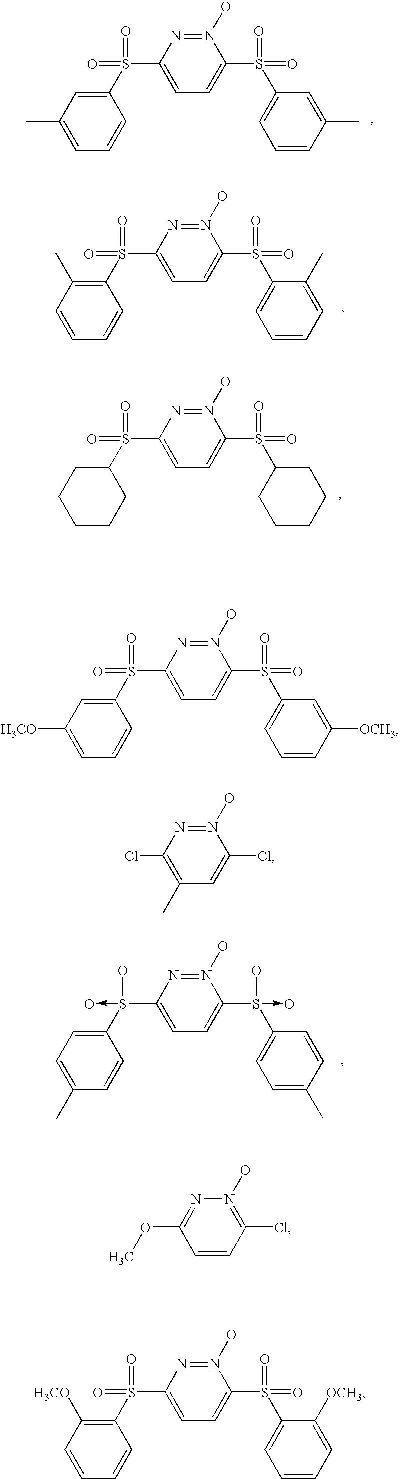

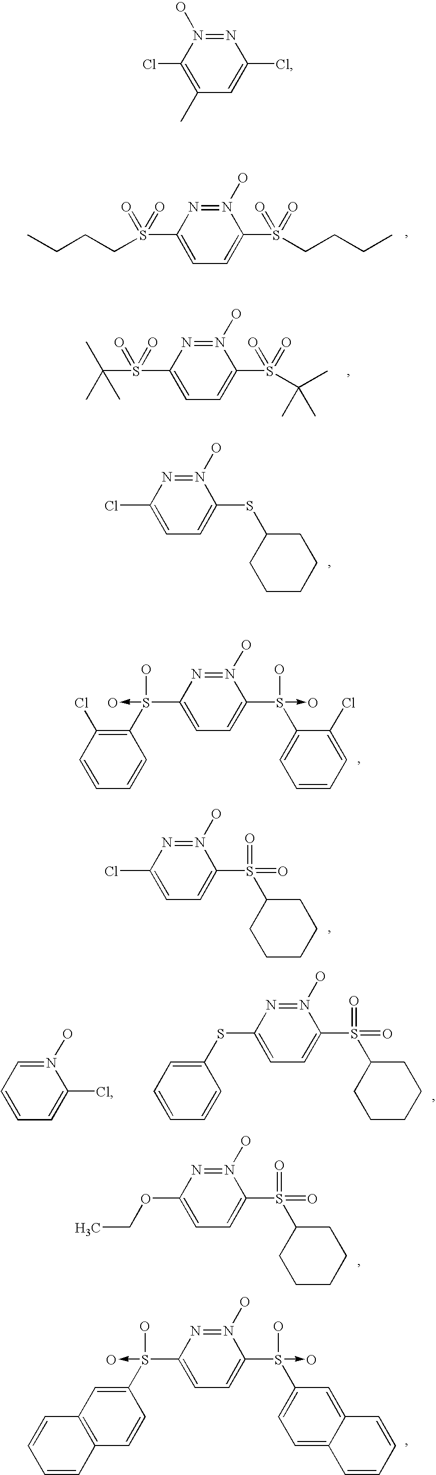

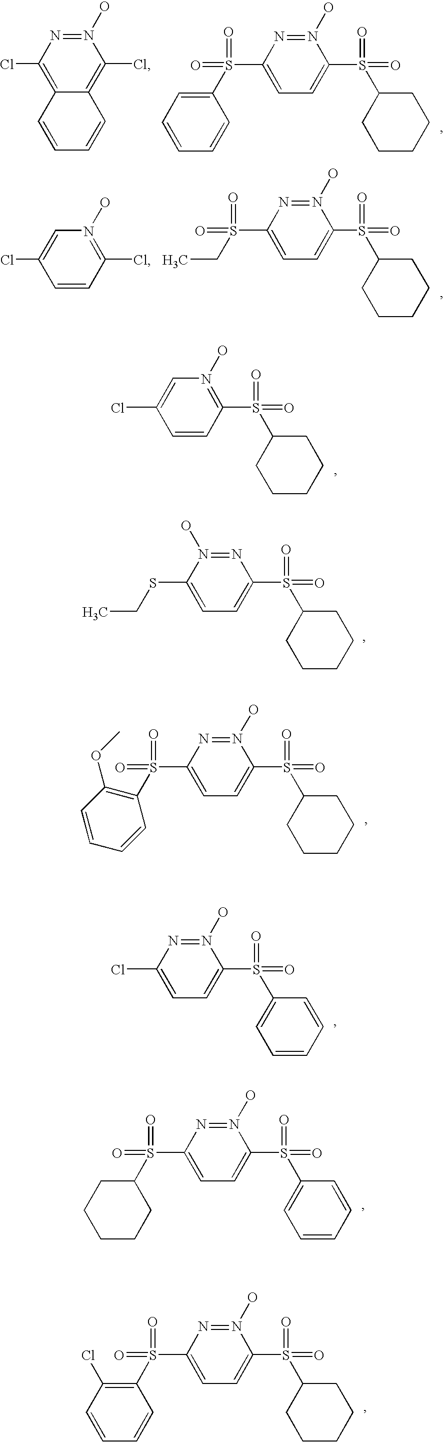

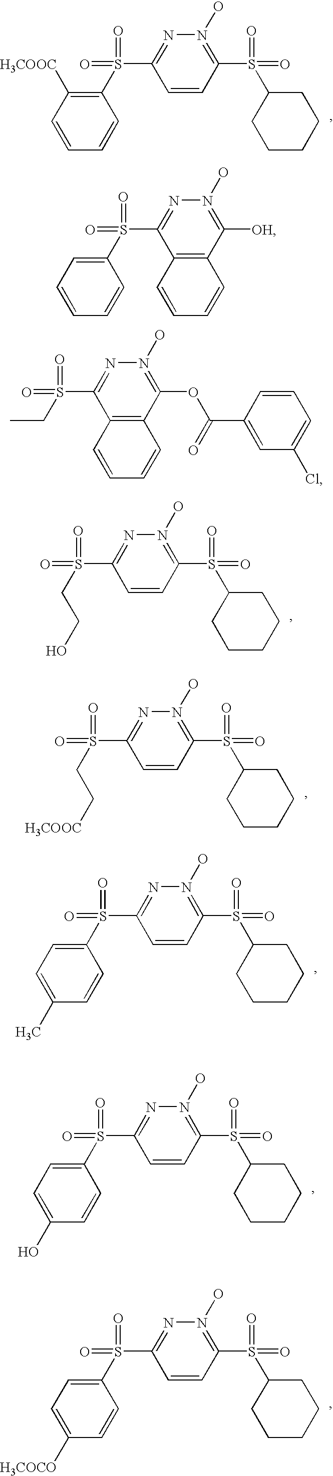

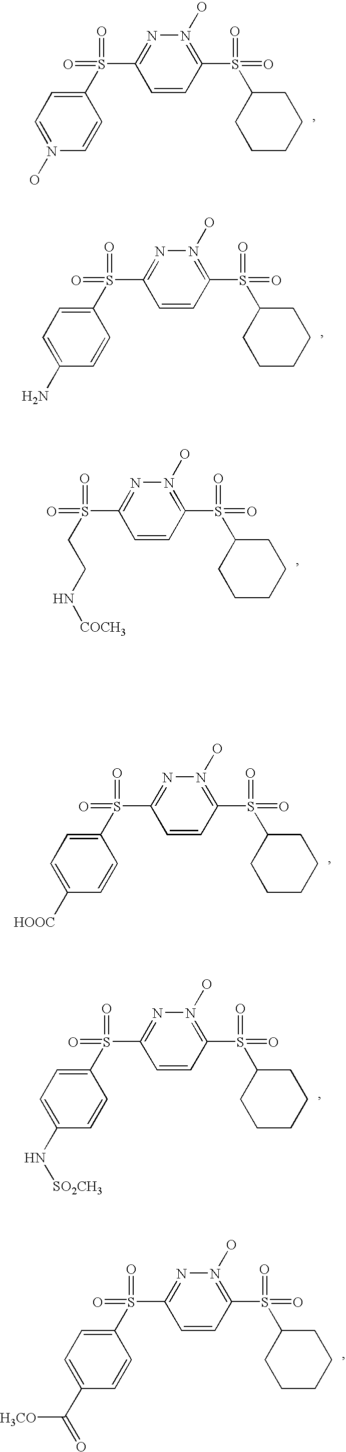

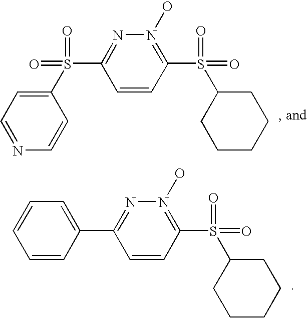

87. An article of manufacture, comprising packaging material, a compound of formula 8: 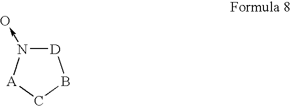
or a pharmaceutically acceptable derivative thereof, wherein
A is selected from 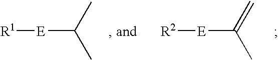
B is selected from 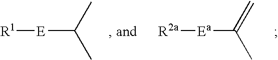
C is selected from 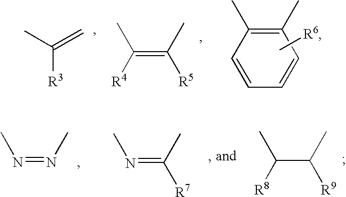
D is selected from —N—, —NO—, —NR¹⁰, —CR¹¹R¹²—,—CR¹³—, —S—, —SO—, and —SO₂—;
E and E^{a} are each independently selected from single bond, —CR¹⁴R¹⁵, —NR¹⁶, —O—, —S—, —SO—, and —SO₂;
R¹ to R⁵, R⁷ to R¹⁶, and R^{2a} are each independently selected from hydrogen, C1-C10 alkyl, C3-C10 cycloalkyl, C1-C10 acyl, C1-C10 alkoxy, C1-C10 alkoxyalkyl, amino, C1-C10 aminoalkyl, C1-C10 alkylaminoalkyl, hydroxyl, C1-C10 hydroxyalkyl, carboxy, C1-C10 carboxyalkyl, C1-C10 alkoxycarbonyl, C1-C10 alkoxycarbonylalkyl, halogen, mono- or polyhaloalkyl, mono- or polyhaloalkoxy, cyano, nitro, mercapto, C1-C10 mercaptoalkyl, C1-C10 thioalkyl, C1-C10 alkylthioalkyl, C1-C10 sulfonylalkyl, C1-C10 alkylsulfonylalkyl, C1-C10 alkylcarbonylaminoC1-C10alkyl, C₃-C₂₀ heterocyclyl, C₃-C₂₀ heterocyclylalkyl, C₅-C₂₀ aralkyl, C₅-C₂₀ aryl, C₅-C₂₀ heteroaralkyl and C₅-C₂₀ heteroaryl, wherein the aryl and heteroaryl groups are optionally substituted with one or more halogen, trihaloalkyl, cyano, nitro, carboxy, alkoxycarbonyl, hydroxyl, alkoxy, acyloxyl, amino, alkylamino, acylamino, mercapto, or alkylthio; and
R⁶ is selected from hydrogen; electron donating groups such as C1-C10 alkyl, C3-C10 cycloalkyl, hydroxyl, C1-C10 alkoxy, amino, C1-C10 acylamino, mercapto or C1-C10 alkylthio; and electron withdrawing groups such as C1-C10 acyl, halo, mono- or polyhaloalkyl, cyano, nitro, carboxy, C1-C10 alkoxycarbonyl, C1-C10 alkylsulfonyl, C5-C10 aryl, C1-C10 alkoxyalkyl, C1-C10 aminoalkyl, C1-C10 alkylaminoalkyl, C1-C10 hydroxyalkyl, C1-C10 carboxyalkyl, C1-C10 alkoxycarbonylalkyl, C1-C10 mercaptoalkyl, C1-C10 alkylthioalkyl, C1-C10 sulfonylalkyl, and C1-C10 alkylsulfonylalkyl,
which is effective for modulating the activity of Bcl-2 protein or for treatment, prevention or amelioration of one or more symptoms of Bcl-2 protein mediated diseases or disorders, or diseases or disorders in which Bcl-2 protein is implicated, within the packaging material, and a label that indicates that the compound or pharmaceutically acceptable derivative thereof is used for modulating the activity of a Bcl-2 protein or for treatment, prevention or amelioration of one or more symptoms of Bcl-2 protein mediated diseases or disorders, or diseases or disorders in which Bcl-2 protein is implicated.

88. A method of treating, preventing, or ameliorating the symptoms of a disease or disorder that is modulated or otherwise affected by Bcl-2 protein or in which Bcl-2 protein is implicated, comprising administering to a subject in need thereof an effective amount of a compound of claim 1.

89. The method of claim 88, wherein the disease or disorder is a Bcl-2 or Bcl-XL mediated disease or disorder.

90. The method of claim 88, wherein the disease or disorder is characterized by overexpression of a Bcl-2 or Bcl-XL protein.

91. The method of claim 88, wherein the disease or disorder is selected from cancers, tumors, hyperproliferative diseases, acquired immune deficiency syndrome, degenerative conditions, and vascular diseases.

92. The method of claim 90, wherein the cancer is selected from B-cell lymphoma including B-cell lymphoma-2, B-cell leukemia, skin cancer, pancreatic cancer, ovarian cancer, liver cancer, bladder cancer, adrenal carcinoma, breast cancer, prostate cancer, and colorectal cancer.

93. A method of treating, preventing, or ameliorating the symptoms of a disease or disorder that is modulated or otherwise affected by Bcl-2 protein or in which Bcl-2 protein is implicated, comprising administering to a subject in need thereof an effective amount of a compound of claim 79.

94. A method of modulating the activity of a Bcl-2 protein, comprising administering to a subject in need thereof an effective amount of a compound of claim 1.

95. The method of claim 94, wherein the Bcl-2 protein is selected from anti-apoptotic Bcl-2 protein, Bcl-2 and Bcl-X_{L}.

96. A method of antagonizing Bcl-2 protein, comprising administering to a subject in need thereof an effective amount of a compound of claim 1.

97. The method of claim 95, wherein the Bcl-2 protein is selected from anti-apoptotic Bcl-2 protein, Bcl-2 and BCl-X_{L}.

98. A method of altering the interaction of an anti-apoptotic Bcl-2 protein, comprising administering to a subject in need thereof an effective amount of a compound of claim 1.

99. The method of claim 95, wherein the Bcl-2 protein is selected from anti-apoptotic Bcl-2 protein, Bcl-2 and BCl-X_{L}.

100. A method of inducing apoptosis, comprising administering to a subject in need thereof an effective amount of a compound of claim 1.

101. A method of modulating the activity of a Bcl-2 protein, comprising administering to a subject in need thereof an effective amount of a compound of claim 79.

102. A method of antagonizing Bcl-2 protein, comprising administering to a subject in need thereof an effective amount of a compound of claim 79.

103. A method of altering the interaction of an anti-apoptotic Bcl-2 protein, comprising administering to a subject in need thereof an effective amount of a compound of claim 79.

104. A method of inducing apoptosis, comprising administering to a subject in need thereof an effective amount of a compound of claim 79.
